SEQUENCE LISTING (4 sequences)

SEQ ID NO: 1 - DNA, 431 bp, Aulosira
  misc_feature (431)..(434): Consensus
cgaaagcctgacggagcaataccgcgtgagggaggaaggctcttgggtcgtaaacctcttttctcagggaagaacaaaatgacggtacctgaggaataagcatcggctaactccgtgccagcagccgcggtaatacggaggatgcaagcgttatccggaatgattgggcgtaagcgtccgcaggtggctatgtaagtctgctgttaaagagtgaggctcaacctcataaaagcagtggaaactacatggctagagtgcgttcggggcagagggaattcctggtgtagcggtgaaatgcgtagagatcaggaagaacaccggtggcgaaagcgctctgctaggccgcaactgacactgagggacgaaagctaggggagcgaatgggattagataccccagtagtcctgagactccaaggcacacaggggata

SEQ ID NO: 2 - DNA, 401 bp, Anabaena
  misc_feature (75)..(77): consensus
cgaaagcctgacggagcaataccgcgtgagggaggaaggctcttgggtcgtaaacctcttttctcagggaagaaaaatgacggtacctgaggaataagcatcggctaactccgtgccagcagccgcggtaatacggaggatgcaagcgttatccggaatgattgggcgtaagcgtccgcaggtggccatgtaagtctgctgttaaagagtcatgcttaacatgataaaagcagtggaaactacagagctagagtacgttcggggcagagggaattcctggtgtagcggtgaaatgcgtagatatcaggaagaacaccggtggcgaaagcgctctgctaggccgtaactgacactgagggacgaaagctaggggagcgaatgggattagataccccagtagt

SEQ ID NO: 3 - DNA, 378 bp, Nostoc
cgaaagcctgacggagcaataccgcgtgagggaggaaggctcttgggttgtaaacctcttttctcagggaataaaatgaaggtacctgaggaatcagcatcggctaactccgtgccagcagccgcggtaatacggaggatgcaagcgttatccggaatgattgggcgtaagcgtccgcaggtggcgatgtaagtctgctgttaaagagtgaggcttaaacctcataaagcagtggaaactacatcgctagagtgcgttcggggcagagggaattcctggtgtagcggtgaaatgcgtagagatcaggaagaacaccggtggcgaaggcgctctgctaggccgcaactgacactgagggacgaaagctaggggagcgaa

SEQ ID NO: 4 - DNA, 404 bp, Tolypothrix
cgaaagcctgacggagcaataccgcgtgagggaggaaggctcttgggttgtaaacctcttttctcagggaagaattaaatgacggtacctgaggaataagcatcggctaactccgtgccagcagccgcggtaatacggaggatgcaagcgttatccggaatgattgggcgtaaagcgtccgcaggtggctatgtaagtctgctgttaaagaatctggctcaaccagataaaggcagtggaaactacatggctagagtgcgttcggggcagaggggaatttcctggtgtagcggtgaaatgcgtagagatcaggagaacaccggtgcgaaagcgctctgctaggccgcaactgacactgagggacgaaagctaggggagcgaatgggattagataccccagtagt

(19) United States
(12) Patent Application Publication
(10) Pub. No.: US 2019/0090410 A1
(43) Pub. Date: Mar. 28, 2019

(54) CYANOBACTERIUM TREATED MONOCOT PLANT SEEDS AND RELATED METHODS

(71) Applicant: Terra Biologics LLC, St. Louis, MO (US)

(72) Inventors: Michael Ott, Iowa City, IA (US); Lawrence E. Page, St. Louis, MO (US)

(21) Appl. No.: 16/139,286

(22) Filed: Sep. 24, 2018

Related U.S. Application Data
(60) Provisional application No. 62/563,062, filed on Sep. 25, 2017.

(51) Int. Cl. A01C 1/06; A01H 5/10; A01N 63/02; C12N 1/20

(52) U.S. Cl. CPC A01C 1/06; A01H 5/10; C12R 1/01; C12N 1/20; A01N 63/02

(57) ABSTRACT

Monocot seeds treated with various cyanobacterium compositions, methods of making the treated seeds, and methods of using the seeds to improve monocot plant growth, biomass, nitrogen uptake, nutrient uptake, lateral root growth, lateral root hair growth, vertical root growth, root branching and/or yield are provided.

### CROSS REFERENCE TO RELATED APPLICATIONS

This application claims the benefit of U.S. Provisional Patent Application Ser. No. 62/563,062 filed on Sep. 25, 2017, the disclosure of which is incorporated herein by reference in its entirety.

### STATEMENT REGARDING FEDERALLY SPONSORED RESEARCH OR DEVELOPMENT

Not applicable

### THE NAMES OF PARTIES TO A JOINT RESEARCH AGREEMENT

Not applicable

### REFERENCE TO A SEQUENCE LISTING, TABLE, OR COMPUTER PROGRAM LISTING APPENDIX SUBMITTED ON A COMPACT DISC AND AN INCORPORATION-BY-REFERENCE OF THE MATERIAL ON A COMPACT DISC

The instant application contains a Sequence Listing as a text file, which is entitled “54886-154379-SEQ-LIST-ST25.txt,” as created on Apr. 5, 2016, and is 2,942 bytes in size. This sequence listing was submitted via EFS-Web in ASCII format on Sep. 25, 2017, and is hereby incorporated by reference in its entirety.

### STATEMENT REGARDING PRIOR DISCLOSURES BY THE INVENTOR OR JOINT INVENTORS

The following disclosure(s) are submitted under 35 U.S.C. § 102(b): WO 2016/164819 published Oct. 13, 2016 to inventors Michael Ott and Lawrence E. Page, and assigned to Terra Biologics LLC.

### FIELD OF THE INVENTION

Compositions are provided and can be used, for example, in coating seeds or other particles or in methods for improved grain yield, nitrogen uptake, nutrient uptake, lateral root growth, lateral root hair growth, vertical root growth, root branching, and biomass, optionally under nitrogen limiting growth conditions.

### BACKGROUND OF THE INVENTION

Cyanobacteria are photosynthetic prokaryotes of the phylum Cyanobacteria. Although Cyanobacteria have been referred to as “blue-green algae,” it is now recognized that Cyanobacteria should not be considered “algae” as they are prokaryotes rather than eukaryotes.

Treatment of various monocot plants with certain Cyanobacteria to improve monocot plant growth has been reported. Treatment of soil with a Nostoc strain soon after seed germination has been reported to improve maize growth and nitrogen uptake (Maqubela et al., Plant Soil Plant and Soil 315: 79-92, 2009). However, Maqubela et al. also report that the “high rate of application cannot be recommended for field scale application as the amount of inoculum required would be unrealistically high” (Maqubela et al. at page 91). Treatment of rice paddy fields with a mixture of Anabaena, Nostoc, Aulosira and Tolypothrix to improve rice yield has also been reported (Mishra and Pabbi, Resonance 9(6): 6-10, 20040). Although improved methods of producing the cyanobacterial inoculum are reported by Mishra and Pabbi, they report that “the effects of inoculation” are inconsistent and that the best results were obtained when the inocula are produced from local stocks and used with a low level of nitrogenous fertilizer (Mishra and Pabbi at page 9).

Field trials performed using various Cyanobacteria treatments on rice have shown that certain treatments can increase yield by 22% and total nitrogen uptake by 59% (International Patent Application Publication WO 2016/164819). Yield improvements over controls for the Cyanobacteria treatments were most pronounced when the rice was grown without added fertilizer.

Greenhouse experiments performed using various Cyanobacteria treatments on cotton have also shown that certain Cyanobacteria treatments performed under certain soil conditions improved biomass production in comparison to untreated controls when no nitrogen fertilizer is provided (International Patent Application Publication WO 2016/164813).

### BRIEF SUMMARY OF THE INVENTION

A treated monocot plant seed is provided, the seed comprising a solid coating on at least a portion of an outer surface of the seed. The coating comprises cyanobacteria comprised of an Aulosira species and a Tolypothrix species, wherein either:

(1) the coating is free of an agriculturally acceptable adjuvant and/or an agriculturally acceptable excipient; or

(2) the coating further comprises the adjuvant and/or the excipient in an amount less than 0.09 gram per gram of seed; or

(3) grain yield resulting from the treated monocot plant seed other than rice is at least 2.0% greater than grain yield resulting from the same monocot plant seed that is not treated with the coating when grown under the following greenhouse conditions:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) for rice, permanent flood is established at 4-leaf growth stage;

(c) temperature maintained at 25-28° C., 50-70% relative humidity and natural light supplemented with fluorescent light at 35 μmol m⁻² s⁻² photosynthetically active radiation to provide a 14 h photoperiod; and

(d) plant grown to 50% heading then entire above-ground biomass is collected and dried, and total weight and nitrogen concentration can be measured; or

(4) grain yield resulting from the treated rice seed is at least 10.0% greater than grain yield resulting from the same rice seed that is not treated with the coating when grown under the greenhouse conditions; or

(5) the cyanobacteria consists essentially of the Aulosira species and the Tolypothrix species; or

(6) the coating consists essentially of the Aulosira species and the Tolypothrix species; or

(7) the coating comprises the Aulosira species and the Tolypothrix species in a weight ratio of about 5:1 to about 1:5.

Also provided is a method of obtaining a plurality of monocot crop plants with improved grain yield, nitrogen uptake, nutrient uptake, biomass, lateral root growth, lateral root hair growth, vertical root growth, or root branching, the method comprising: distributing the treated monocot plant seed on a plot of land and cultivating plants grown from the seed on the plot.

A method of obtaining a monocot plant seed that provides for improved growth in a monocot plant obtained from the seed is also provided. The method comprises: applying a composition comprising the cyanobacteria to the at least a portion of an outer surface of the seed, and lyophilizing or drying the composition to form the solid coating and obtain the treated monocot plant seed.

Also provided is a method of improving grain yield, nitrogen uptake, nutrient uptake, biomass, lateral root growth, lateral root hair growth, vertical root growth, or root branching in a monocot plant, the method comprising:

exposing the monocot plant seed to an effective amount of a composition comprising cyanobacteria comprised of an Aulosira species and a Tolypothrix species, wherein either:

(1) the composition is free of an agriculturally acceptable adjuvant and/or an agriculturally acceptable excipient; or

(2) the composition further comprises the adjuvant and/or the excipient in an amount less than 0.4 kg/hectare; or

(3) the cyanobacteria consists essentially of the Aulosira species and the Tolypothrix species; or

(4) the composition consists essentially of the Aulosira species and the Tolypothrix species; or

(5) the composition comprises the Aulosira species and the Tolypothrix species in a weight ratio of about 5:1 to about 1:5; and

allowing the monocot plant seed to germinate;

wherein either:

(A) grain yield resulting from the treated monocot plant seed other than rice is at least 2.0% greater than grain yield resulting from the same monocot plant seed that is not exposed to the composition when grown under greenhouse conditions; or

(B) grain yield resulting from the treated rice seed is at least 10.0% greater than grain yield resulting from the same rice seed that is not exposed to the composition when grown under the greenhouse conditions; or

(C) nitrogen uptake resulting from the treated monocot plant seed is at least 5% greater than nitrogen uptake resulting from the same monocot plant seed that is not exposed to the composition when grown under the greenhouse conditions; or

(D) biomass resulting from the treated monocot plant seed is at least 2% greater than biomass resulting from the same monocot plant seed that is not exposed to the composition when grown under the greenhouse conditions; or

(E) lateral root growth, lateral root hair growth, vertical root growth, or root branching resulting from the treated monocot plant seed exhibits increased branching as compared to lateral root growth, lateral root hair growth, vertical root growth, or root branching resulting from the same monocot plant seed that is not exposed to the composition when grown under the greenhouse conditions; or

(F) nutrient uptake resulting from the treated monocot plant seed is at least 2% greater than nutrient uptake resulting from the same monocot plant seed that is not exposed to the composition when grown under the greenhouse conditions;

wherein the greenhouse conditions are as follows:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) for rice, permanent flood is established at 4-leaf growth stage;

(c) temperature maintained at 25-28° C., 50-70% relative humidity and natural light supplemented with fluorescent light at 35 μmol m⁻² s⁻² photosynthetically active radiation to provide a 14 h photoperiod; and

(d) plant grown to 50% heading then entire above-ground biomass is collected and dried, and total weight and nitrogen concentration can be measured.

Also provided is a treated particle comprising a solid coating on at least a portion of an outer surface of the particle, the coating comprising a cyanobacterium comprised of an Aulosira species, and the particle comprising a fertilizer granule or a biochar particle.

Also provided is a method of binding an agriculturally acceptable adjuvant and/or an agriculturally acceptable excipient to a particle, the method comprising: applying a composition comprising the cyanobacteria and the adjuvant and/or excipient to the at least a portion of an outer surface of the particle and lyophilizing or drying the composition to form the solid coating and obtain the treated particle as described herein.

Also provided is a method of obtaining a plurality of plants, the method comprising: distributing the treated particles on a plot of land; planting the plot with seeds or plants; and cultivating plants grown on the plot. The method can further comprise harvesting the cultivated plants.

Also provided is a method of obtaining a treated particle comprising: applying a composition comprising the cyanobacteria to the at least a portion of an outer surface of the particle and lyophilizing or drying the composition to form the solid coating and obtain the treated particle.

Other objects and features will be in part apparent and in part pointed out hereinafter.

### BRIEF DESCRIPTION OF THE SEVERAL VIEWS OF THE DRAWING

FIG. 1 shows the effects of treating corn seeds with cyanobacteria. Root hair formation was visualized using digital photography. Seeds on the left were not treated with cyanobacteria, while seeds on the right were treated with 4 mg of cyanobacteria. An increase in lateral root and lateral root hair formation was observed in the cyanobacteria-treated seeds. Images were taken on day 6 of the germination test.

FIG. 2 shows the effects of a topical application of cyanobacteria on rice biomass accumulation at half-heading. Statistically significant biomass accumulation (Y-axis) was observed across all fertilizer levels (X-axis) tested. The cyanobacteria application rate (BSC) is represented by the bar shading and accumulation rate improvement over control (OX) is listed on the graph over each bar as a percentage. No statistical significance was observed between the cyanobacteria application rates, meaning both rates were equally effective in improving accumulation.

FIG. 3 shows the response of two corn varieties (hybrid germplasms) to three cyanobacteria treatments (A, B, C) under four fertilizer levels is depicted. Statistically significant improvements relative to the control that are positive are above the baseline and shown as open bars. Statistically significant negative effects relative to the control are below the baseline and shown as black bars. Effects relative to the control that were not statistically significant are shown with grey bars Biomass increased in week 3 and week 4 of growth in multiple treatments and multiple fertilizer levels.

FIG. 4 shows the response of corn germplasms G1 and G2 to three cyanobacteria treatments under two fertilizer levels. Statistically significant improvements relative to the control that are positive are denoted by open (white) squares. Statistically significant negative effects relative to the control are denoted by black squares. All grey squares showed no statistically significant effect over control. Germplasm 2 (G2) showed 27 positive responses compared to 13 negative responses.

### DETAILED DESCRIPTION OF THE INVENTION

It has been discovered that seeds can be coated with the minimal amount of cyanobacteria which can provide a commercially significant increase in yield, nitrogen uptake, nutrient uptake, biomass, lateral root growth, lateral root hair growth, vertical root growth, and/or root branching. The seed coating need not contain any ingredients other than cyanobacteria if desired so that the coating can be comprised of entirely biologically active ingredients. The cyanobacteria is sufficiently tacky to adhere to the seed without the need for other tackifiers or other typical coating ingredients.

Provided herein are monocot plant seeds treated with various cyanobacterial compositions that provide improved monocot plant growth characteristics, methods of making the treated seeds, and methods of using the seeds. Such improved growth characteristics conferred by the seed treatments include, but are not limited to, improved nitrogen uptake, increased nutrient uptake, increased lateral root growth, increased lateral root hair growth, increased vertical root growth, increased root branching and increased yield under nitrogen limiting growth conditions. Also provided are monocot plant seeds, methods of making, and methods of use where the seeds are treated with combinations of cyanobacterium species that can effect more pronounced improvements in the aforementioned growth characteristics when compared to untreated seeds or seeds treated with other combinations of cyanobacterium species.

A treated monocot plant seed is provided, the seed comprising a solid coating on at least a portion of an outer surface of the seed. The coating comprises cyanobacteria comprised of an Aulosira species and a Tolypothrix species, wherein either:

(1) the coating is free of an agriculturally acceptable adjuvant and/or an agriculturally acceptable excipient; or

(2) the coating further comprises the adjuvant and/or the excipient in an amount less than 0.09 gram per gram of seed; or

(3) grain yield resulting from the treated monocot plant seed other than rice is at least 2.0% greater than grain yield resulting from the same monocot plant seed that is not treated with the coating when grown under the following greenhouse conditions:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) for rice, permanent flood is established at 4-leaf growth stage;

(c) temperature maintained at 25-28° C., 50-70% relative humidity and natural light supplemented with fluorescent light at 35 μmol m⁻² s⁻² photosynthetically active radiation to provide a 14 h photoperiod; and

(d) plant grown to 50% heading then entire above-ground biomass is collected and dried, and total weight and nitrogen concentration can be measured; or (4) grain yield resulting from the treated rice seed is at least 10.0% greater than grain yield resulting from the same rice seed that is not treated with the coating when grown under the greenhouse conditions; or

(5) the cyanobacteria consists essentially of the Aulosira species and the Tolypothrix species; or

(6) the coating consists essentially of the Aulosira species and the Tolypothrix species; or

(7) the coating comprises the Aulosira species and the Tolypothrix species in a weight ratio of about 5:1 to about 1:5.

The coating can be free of an agriculturally acceptable adjuvant or an agriculturally acceptable excipient, or can be free of both the adjuvant and excipient.

If an amount of the adjuvant or excipient is present in the coating, the coating comprises the adjuvant and/or the excipient in an amount of at least 0.001 gram per gram of seed. The adjuvant or excipient can be in an amount less than 0.08, 0.07, 0.06, 0.05, 0.04, 0.03, 0.02 or 0.01 gram per gram of seed.

The cyanobacteria can consist essentially of the Aulosira species and the Tolypothrix species.

The coating can consist essentially of the Aulosira species and the Tolypothrix species.

Also provided herein are treated monocot plant seeds that have been at least partially coated with a composition comprising: (i) at least one cyanobacterium species; and (ii) an agriculturally acceptable adjuvant, an agriculturally acceptable excipient, or a combination thereof.

Compositions used to treat monocot plant seeds can comprise (i) at least one cyanobacterium species; and (ii) an agriculturally acceptable adjuvant, an agriculturally acceptable excipient, or a combination thereof.

Cyanobacteria used in the compositions and coatings provided herein include, but are not limited to, cyanobacteria in the order Nostocales.

The composition or coating can comprise one, two, three, or four cyanobacterium species that are members of the family Nostocaceae and/or at least one cyanobacterium species that is a member of the family Microchaetaceae.

The member or members of the family Nostocaceae can comprise a Nostoc sp., an Aulosira sp., and/or an Anabaena sp., and the member of the family Microchaetaceae can comprise a Tolypothrix sp.

Non-limiting examples of a Nostoc sp., an Aulosira sp., an Anabaena sp., and a Tolypothrix sp. and cultures comprising the same that can be used in the compositions or coatings are provided in Table 1.

TABLE 1
Cyanobacterium species

| | | | Geographic Origin |
|---|---|---|---|
| Genus | Species | Source ¹ | of Source Organism |
| Nostoc | commune | Nostoc commune | Austin, Texas; USA |
| | | UTEX B 1621 | |
| Aulosira | bohemensis | Aulosira bohemensis | Dlouha Ves, South |
| | | UTEX B 2947 | Bohemia; Czech |
| | | | Republic |
| Anabaena | cylindrica | Anabaena cylindrica | England |
| | | UTEX B 1611 | |
| Tolypothrix | distorta | Tolypothrix distorta | Utrecht, Utrecht; |
| | | UTEX 424 | Netherlands |

¹ All strains with a “UTEX” designation are publicly available through the University of Texas at Austin UTEX Culture Collection of Algae, 1 University Station A6700, Austin, TX, USA or via the internet on the world wide web site “utex.org.”

The member or members of the family Nostocaceae can comprise a Nostoc commune UTEX B 1621 culture, an Aulosira bohemensis UTEX B 2947 culture, an Anabaena cylindrica UTEX B 1611 culture, isolate(s) therefrom, or a variant thereof and the member or members of the family Microchaetaceae can comprise a Tolypothrix distorta UTEX 424 culture, isolate(s) therefrom, or a variant thereof.

Cyanobacterium that can be used in the compositions, coatings and methods described herein can also be identified by the sequence of the gene encoding the 16S RNA. The cyanobacteria that are used can be characterized by having a gene encoding a 16S RNA that has at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4. For example, the cyanobacterium species can be characterized by having a gene encoding a 16S RNA that has at least 95%, 97%, 98%, 99%, 99.5%, 99.7%, 99.9%, or 100% sequence identity across the entire length of SEQ ID NO:1 and/or SEQ ID NO:4.

Useful 16S RNA sequences are provided in Table 2.

TABLE 2
Cyanobacterium 16S RNA sequences

| | SEQ | |
|---|---|---|
| | ID | Consensus Sequence |
| Genus | NO: | of gene encoding 16S RNA¹ |
| Aulosira | 1 | CGAAAGCCTGACGGAGCAATACCGC |
| | | GTGAGGGAGGAAGGCTCTTGGGTCG |
| | | TAAACCTCTTTTCTCAGGGAAGAAC |
| | | AAAATGACGGTACCTGAGGAATAAG |
| | | CATCGGCTAACTCCGTGCCAGCAGC |
| | | CGCGGTAATACGGAGGATGCAAGCG |
| | | TTATCCGGAATGATTGGGCGTAAGC |
| | | GTCCGCAGGTGGCTATGTAAGTCTG |
| | | CTGTTAAAGAGTGAGGCTCAACCTC |
| | | ATAAAAGCAGTGGAAACTACATGGC |
| | | TAGAGTGCGTTCGGGGCAGAGGGAA |
| | | TTCCTGGTGTAGCGGTGAAATGCGT |
| | | AGAGATCAGGAAGAACACCGGTGGC |
| | | GAAAGCGCTCTGCTAGGCCGCAACT |
| | | GACACTGAGGGACGAAAGCTAGGGG |
| | | AGCGAATGGGATTAGATACCCCAGT |
| | | AGTCCTGAGACTCCAAGGCAcacaG |
| | | GGGATA |
| Anabaena | 2 | CGAAAGCCTGACGGAGCAATACCGC |
| | | GTGAGGGAGGAAGGCTCTTGGGTCG |
| | | TAAACCTCTTTTCTCAGGGAAGAAa |
| | | aaTGACGGTACCTGAGGAATAAGCA |
| | | TCGGCTAACTCCGTGCCAGCAGCCG |
| | | CGGTAATACGGAGGATGCAAGCGTT |
| | | ATCCGGAATGATTGGGCGTAAGCGT |
| | | CCGCAGGTGGCCATGTAAGTCTGCT |
| | | GTTAAAGAGTCATGCTTAACATGAT |
| | | AAAAGCAGTGGAAACTACAGAGCTA |
| | | GAGTACGTTCGGGGCAGAGGGAATT |
| | | CCTGGTGTAGCGGTGAAATGCGTAG |
| | | ATATCAGGAAGAACACCGGTGGCGA |
| | | AAGCGCTCTGCTAGGCCGTAACTGA |
| | | CACTGAGGGACGAAAGCTAGGGGAG |
| | | CGAATGGGATTAGATACCCCAGTAG |
| | | T |
| Nostoc | 3 | CGAAAGCCTGACGGAGCAATACCGC |
| | | GTGAGGGAGGAAGGCTCTTGGGTTG |
| | | TAAACCTCTTTTCTCAGGGAATAAA |
| | | ATGAAGGTACCTGAGGAATCAGCAT |
| | | CGGCTAACTCCGTGCCAGCAGCCGC |
| | | GGTAATACGGAGGATGCAAGCGTTA |
| | | TCCGGAATGATTGGGCGTAAGCGTC |
| | | CGCAGGTGGCGATGTAAGTCTGCTG |
| | | TTAAAGAGTGAGGCTTAAACCTCAT |
| | | AAAGCAGTGGAAACTACATCGCTAG |
| | | AGTGCGTTCGGGGCAGAGGGAATTC |
| | | CTGGTGTAGCGGTGAAATGCGTAGA |
| | | GATCAGGAAGAACACCGGTGGCGAA |
| | | GGCGCTCTGCTAGGCCGCAACTGAC |
| | | ACTGAGGGACGAAAGCTAGGGGAGC |
| | | GAA |
| Tolypothrix | 4 | CGAAAGCCTGACGGAGCAATACCGC |
| | | GTGAGGGAGGAAGGCTCTTGGGTTG |
| | | TAAACCTCTTTTCTCAGGGAAGAAT |
| | | TAAATGACGGTACCTGAGGAATAAG |
| | | CATCGGCTAACTCCGTGCCAGCAGC |
| | | CGCGGTAATACGGAGGATGCAAGCG |
| | | TTATCCGGAATGATTGGGCGTAAAG |
| | | CGTCCGCAGGTGGCTATGTAAGTCT |
| | | GCTGTTAAAGAATCTGGCTCAACCA |
| | | GATAAAGGCAGTGGAAACTACATGG |
| | | CTAGAGTGCGTTCGGGGCAGAGGGG |
| | | AATTTCCTGGTGTAGCGGTGAAATG |
| | | CGTAGAGATCAGGAGAACACCGGTG |
| | | CGAAAGCGCTCTGCTAGGCCGCAAC |
| | | TGACACTGAGGGACGAAAGCTAGGG |
| | | GAGCGAATGGGATTAGATACCCCAG |
| | | TAGT |

¹16S consensus sequences were generated by harvesting liquid cyanobacterial cultures, amplifying the 16S ribosomal region by PCR, gel-purifying the 16S band, and sequencing the resulting PCR product. Sequences are made up of many reads that were combined to generate a consensus sequence. Lower-case letters in the published sequences denote locations where variation was found in the base at that position such that it reduced confidence to below the set threshold.

The member or members of the family Nostocaceae can be characterized by having a gene encoding a 16S RNA that has at least 95%, 97%, 98%, 99%, 99.5%, 99.7%, 99.9%, or 100% sequence identity across the entire length of SEQ ID NO:1, SEQ ID NO:2, or SEQ ID NO:3, and the member or members of the family Microchaetaceae can be characterized by having a gene encoding a 16S RNA that has at least 95%, 97%, 98%, 99%, 99.5%, 99.7%, 99.9%, or 100% sequence identity across the entire length of SEQ ID NO:4.

The member or members of the family Nostocaceae can be characterized by having a gene encoding a 16S RNA that has at least 95%, 97%, 98%, 99%, 99.5%, 99.7%, 99.9%, or 100% sequence identity across the entire length of SEQ ID NO:1 and the member or members of the family Microchaetaceae can be characterized by having a gene encoding a 16S RNA that has at least 95%, 97%, 98%, 99%, 99.5%, 99.7%, 99.9%, or 100% sequence identity across the entire length of SEQ ID NO:4.

The cyanobacteria can be characterized by having a gene encoding a 16S RNA that has at least 95%, 97%, 98%, 99%, 99.5%, 99.7%, 99.9%, or 100% sequence identity across the entire length of: (i) SEQ ID NO:1; (ii) SEQ ID NO:1 and SEQ ID NO:2; (iii) SEQ ID NO:1 and SEQ ID NO:4; (iv) SEQ ID NO:3 and SEQ ID NO:4; (v) SEQ ID NO:1, SEQ ID NO:2, and SEQ ID NO:3; or (vi) SEQ ID NO:4. For example, cyanobacteria can be characterized by having a gene encoding a 16S RNA that has at least 95%, 97%, 98%, 99%, 99.5%, 99.7%, 99.9%, or 100% sequence identity across the entire length of: (i) SEQ ID NO:1; (ii) SEQ ID NO:1 and SEQ ID NO:2; (iii) SEQ ID NO:1 and SEQ ID NO:4; or (iv) SEQ ID NO:3 and SEQ ID NO:4, or (i) SEQ ID NO:1; (ii) SEQ ID NO:1 and SEQ ID NO:2; (iii) SEQ ID NO:1 and SEQ ID NO:4; (iv) SEQ ID NO:1, SEQ ID NO:2, and SEQ ID NO:3; or (v) SEQ ID NO:4.

The cyanobacteria can comprise any one of: (i) Aulosira sp. and Tolypothrix sp.; (ii) Aulosira sp. and Anabaena sp.; (iii) Aulosira sp.; (iv) Aulosira sp.; Anabaena sp.; and Tolypothrix sp.; or (v) Nostoc sp.

The cyanobacteria can comprise any one of: (i) Aulosira bohemensis sp.; and a Tolypothrix distorta; (ii) Aulosira bohemensis; (iii) Aulosira bohemensis and Anabaena cylindrica; (iv) Aulosira bohemensis, Anabaena cylindrical, and Tolypothrix distorta; or (v) Nostoc commune.

The coating or composition comprises the cyanobacterium in an amount from about 0.001 g to about 0.15 gram per gram of seed, such as in an amount less than 0.08, 0.09, 0.10, 0.11, 0.12, 0.13, or 0.14 gram per gram of seed.

A cyanobacterium useful in compositions, coatings and methods provided herein can also be obtained by isolation from terrestrial sources including, but not limited to soil and plants, as well as aquatic sources.

One useful method for isolating cyanobacteria that can be used involves nutrient saturated glass fiber filters in combination with a broad spectrum beta-lactam antibiotic to isolate cyanobacteria (Ferris and Hirsch, Appl. Environ. Microbio, 57(5): 1448-1452, 1991). Additional methods for obtaining cyanobacteria that use phototaxis and scored agar surfaces in the presence of certain antibiotics can also be used (Vaara et al. Appl. Environ. Microbiol. 38(5):1011-4, 1979). Isolation methods including by not limited to those described by the aforementioned citations and references cited therein can also be used to obtain isolates from non-axenic cyanobacteria cultures to obtain cyanobacterium isolates that can be used in the methods, coatings and compositions provided herein. The isolates are obtained from a non-axenic Nostoc commune UTEX B 1621 culture, a non-axenic Aulosira bohemensis UTEX B 2947 culture, a non-axenic Anabaena cylindrica UTEX B 1611 culture, and a non-axenic Tolypothrix distorta UTEX 424 culture.

Variants of the cyanobacteria can also be used in compositions, coatings and methods provided herein. The cyanobacteria variants are variants that have been induced by mutagenesis and selected for one or more useful traits. Mutagenesis techniques include, but are not limited to use of alkylating agents, intercalating agents, transposons, and the like. Cyanobacteria variants can be screened and then selected for useful traits that include, but are not limited to, increased phototaxis, changes in genome copy number, increased protein production, or altered pigment expression. Variants can also be obtained where the cyanobacteria have been genetically transformed with a heterologous transgene. Methods for transforming cyanobacteria that have been disclosed (Chaurasia, et al., J Microbiol Methods. 73(2):133-41, 2008) can be used to obtain such variants.

Seeds treated with compositions or coatings provided herein can be used to improve a variety of monocot plant growth characteristics including, but not limited to, plant biomass, grain yield, lateral root growth, lateral root hair growth, vertical root growth, root branching, nutrient uptake, growth under nitrogen limiting conditions, and/or nitrogen uptake as discussed in more detail below. The improvement in the monocot plant growth characteristic can be in comparison to the growth characteristic of a monocot plant either obtained from an untreated monocot plant seed or a monocot plant that has been treated with a topical application of cyanobacteria. For example, the improvement in the monocot plant growth characteristic can be in comparison to the growth characteristic of either a monocot plant that has been treated with a topical application of a cyanobacterial mixture or a monocot plant obtained from a monocot plant seed treated with a cyanobacterial mixture, the cyanobacterial mixture comprising a Nostoc commune UTEX B 1621 culture, a Aulosira bohemensis UTEX B 2947 culture, a Anabaena cylindrica UTEX B 1611 culture, and a Tolypothrix distorta UTEX 424 culture.

Monocot seeds can be treated with a composition or coating comprising: (i) Aulosira sp., Anabaena sp., and Tolypothrix sp., or (ii) Nostoc sp.

Monocot seeds can be treated with a composition or coating comprising Aulosira sp., Anabaena sp., Tolypothrix sp., and Nostoc sp.

The Aulosira sp., Anabaena sp., Nostoc sp., and/or Tolypothrix sp. used in the composition or coating can be characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4. For example, the Aulosira sp., Anabaena sp., Nostoc sp., and/or Tolypothrix sp. used in the composition or coating can be characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, or SEQ ID NO:4.

The Aulosira species can be characterized by having a gene encoding a 16S RNA that has at least 95%, 97%, 98%, 99%, 99.5%, 99.7%, 99.9%, or 100% sequence identity across the entire length of SEQ ID NO:1 and the Tolypothrix species are characterized by having a gene encoding a 16S RNA that has at least 95%, 97%, 98%, 99%, 99.5%, 99.7%, 99.9%, or 100% sequence identity across the entire length of SEQ ID NO:4.

The Aulosira species can be characterized by having a gene encoding a 16S RNA that has at least 95%, 97%, 98%, 99%, 99.5%, 99.7%, 99.9%, or 100% sequence identity across the entire length of SEQ ID NO:1.

The Tolypothrix species can be characterized by having a gene encoding a 16S RNA that has at least 95%, 97%, 98%, 99%, 99.5%, 99.7%, 99.9%, or 100% sequence identity across the entire length of SEQ ID NO:4.

The Aulosira species can be an Aulosira bohemensis UTEX B 2947 culture or variant thereof.

The Tolypothrix species can be a Tolypothrix distorta UTEX 424 culture or a variant thereof.

Both an Aulosira bohemensis UTEX B 2947 culture or variant thereof and a Tolypothrix distorta UTEX 424 culture or variant thereof can be used in the methods and seed treatments described herein.

The treated monocot plant seeds can be at least partially coated with a composition comprising: an Aulosira species and a Tolypothrix species at a ratio of 3:1 to 1.5:1, respectively; and (ii) an agriculturally acceptable adjuvant, an agriculturally acceptable excipient, or a combination thereof.

Monocot seeds can be treated with a composition or coating comprising: (i) an Aulosira bohemensis sp. and a Tolypothrix distorta sp.; (ii) an Aulosira bohemensis sp.; (iii) an Aulosira bohemensis sp. and an Anabaena cylindrica sp.; (iv) an Aulosira bohemensis sp., an Anabaena cylindrica sp., and a Tolypothrix distorta sp.; or (v) a Nostoc commune sp.

It may be desirable that (i) the cyanobacterium is not associated with the plant seed in nature; (ii) the composition or coating comprises at least two cyanobacterium species that are not associated with the plant seed in nature; (iii) the composition or coating comprises at least two cyanobacterium species that are not associated with one another in nature; or (iv) the composition or coating comprises an exogeneous exopolysaccharide (EPS). For example, it may be desirable that the Aulosira species and the Tolypothrix species are not associated with the monocot plant seed in nature.

The Aulosira species and the Tolypothrix species may not be associated with the plant seed in nature, or the composition or coating may comprise an exogenous exopolysaccharide (EPS).

It may be desirable that the composition or coating does not further comprise an Anabaena species, a Nostoc species, or both an Anabaena species and a Nostoc species.

It may be desirable that the composition or coating does not further comprise Tolypothrix tenuis, Aulosira fertilissima, and a Nostoc species, or Tolypothrix tenuis, Aulosira fertilissima, a Nostoc species, and a Cylindrospermum species.

Representative examples of cyanobacteria that can be used in the compositions, coatings and methods provided herein that are characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4 include, but are not limited to, one or more of the cyanobacteria provided in Table 3. Representative examples of cyanobacteria that can be used in the compositions, coatings and methods provided herein also include, but are not limited to, combinations of the cyanobacteria provided in Tables 1 and 3.

The cyanobacterium used to treat the seed can be heterologous to the monocot seed. The cyanobacterium are heterologous to the seed when they are not detectable in or on a seed that has not been treated with the compositions or coatings provided herein.

TABLE 3
Representative Cyanobacteria having 16S RNA Sequences with
significant sequence identity to SEQ ID NO: 1, 2, 3, and/or 4

| | | | | |
|---|---|---|---|---|
| | 16S sequence | | | |
| | % identity | | | |
| | to specified | | | Culture Collection |
| Cyanobacterium | SEQ ID NO: | NCBI Accession ¹ | Culture Collection² | Location |

Isolates having a 16S RNA encoding gene with at least 95% sequence
identity to Aulosira bohemensis 16S SEQ ID NO: 1

| | | | | |
|---|---|---|---|---|
| Calothrix sp. | 99% | NC_019682.1 | Pasteur | Paris, |
| PCC 7507 | | | Culture | France |
| | | | Collection | |
| Anabaena | 97% | NC_019771.1 | Pasteur | Paris, |
| cylindrica PCC | | | Culture | France |
| 7122 | | | Collection | |
| Cylindrospermum | 97% | NC_019757.1 | Pasteur | Paris, |
| stagnale PCC | | | Culture | France |
| 7417 | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |
| Nodularia | 96% | NZ_CP007203.1 | Culture | Bratislava, |
| spumigena | | | collection of | Slovakia |
| CCY9414 | | | yeasts | |
| Nostoc azollae | 96% | NC_014248.1 | None | N/A |
| 0708 | | | | |
| Anabaena | 96% | NC_007413.1 | American | Manassas, |
| variabilis ATCC | | | Type Culture | Virginia, |
| 29413 | | | Collection | USA |
| Nodularia | 96% | CP007203.2 | Culture | Bratislava, |
| spumigena | | | collection of | Slovakia |
| CCY9414 | | | yeasts | |
| Nostoc sp. PCC | 95% | NC_019676.1 | Pasteur | Paris, |
| 7107 | | | Culture | France |
| | | | Collection | |
| Anabaena sp. 90 | 95% | NC_019427.1 | University of | Helsinki, |
| | | | Helisinki | Finland |
| | | | Cyanobacterial | |
| | | | Culture | |
| | | | Collection | |

Isolates having at least 95% sequence identity to
Anabaena cylindrica 16S SEQ ID NO: 2

| | | | | |
|---|---|---|---|---|
| Anabaena | 97% | NC_019771.1 | Pasteur | Paris, |
| cylindrica PCC | | | Culture | France |
| 7122 | | | Collection | |
| Calothrix sp. | 99% | NC_019682.1 | Pasteur | Paris, |
| PCC 7507 | | | Culture | France |
| | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |
| Cylindrospermum | 97% | NC_019757.1 | Pasteur | Paris, |
| stagnale PCC | | | Culture | France |
| 7417 | | | Collection | |
| Nostoc sp. PCC | 95% | NC_019676.1 | Pasteur | Paris, |
| 7107 | | | Culture | France |
| | | | Collection | |
| Anabaena | 96% | NC_007413.1 | American | Manassas, |
| variabilis ATCC | | | Type Culture | Virginia, |
| 29413 | | | Collection | USA |
| Nodularia | 96% | NZ_CP007203.1 | Culture | Bratislava, |
| spumigena | | | collection of | Slovakia |
| CCY9414 | | | yeasts | |
| Anabaena sp. 90 | 95% | NC_019427.1 | University of | Helsinki, |
| | | | Helsinki | Finland |
| | | | Cyanobacterial | |
| | | | Culture | |
| | | | Collection | |
| Nostoc azollae | 96% | NC_014248.1 | None | N/A |
| 0708 | | | | |
| Nodularia | 96% | CP007203.2 | Culture | Bratislava, |
| spumigena | | | collection of | Slovakia |
| CCY9414 | | | yeasts | |

Isolates having at least 95% sequence identity to
Nostoc commune 16S SEQ ID NO: 3

| | | | | |
|---|---|---|---|---|
| Cylindrospermum | 97% | NC_019757.1 | Pasteur | Paris, |
| stagnale PCC | | | Culture | France |
| 7417 | | | Collection | |
| Calothrix sp. | 99% | NC_019682.1 | Pasteur | Paris, |
| PCC 7507 | | | Culture | France |
| | | | Collection | |
| Nostoc sp. PCC | 95% | NC_019676.1 | Pasteur | Paris, |
| 7107 | | | Culture | France |
| | | | Collection | |
| Nostoc sp. PCC | 96% | NC_003272.1 | Pasteur | Paris, |
| 7120 | | | Culture | France |
| | | | Collection | |
| Anabaena | 96% | NC_007413.1 | American | Manassas, |
| variabilis ATCC | | | Type Culture | Virginia, |
| 29413 | | | Collection | USA |
| Anabaena | 97% | NC_019771.1 | Pasteur | Paris, |
| cylindrica PCC | | | Culture | France |
| 7122 | | | Collection | |
| Nostoc | 97% | NC_010628.1 | Pasteur | Paris, |
| punctiforme PCC | | | Culture | France |
| 73102 | | | Collection | |

Isolates having at least 95% sequence identity to
Tolypothrix distorta 16S SEQ ID NO: 4

| | | | | |
|---|---|---|---|---|
| Nodularia | 96% | NZ_CP007203.1 | Culture | Bratislava, |
| spumigena | | | collection of | Slovakia |
| CCY9414 | | | yeasts | |
| Calothrix sp. | 99% | NC_019682.1 | Pasteur | Paris, |
| PCC 7507 | | | Culture | France |
| | | | Collection | |
| Nodularia | 96% | CP007203.2 | Culture | Bratislava, |
| spumigena | | | collection of | Slovakia |
| CCY9414 | | | yeasts | |
| Cylindrospermum | 97% | NC_019757.1 | Pasteur | Paris, |
| stagnale PCC | | | Culture | France |
| 7417 | | | Collection | |
| Anabaena | 97% | NC_019771.1 | Pasteur | Paris, |
| cylindrica PCC | | | Culture | France |
| 7122 | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |

¹ Sequences of the whole genomes or a selected chromosome of the indicated strains that contain the 16S RNA encoding sequences can be obtained from the National Center Biotechnology Information National Center for Biotechnology Information (NCBI) with the indicated accession numbers via the internet at the world wide web site “ncbi.nlm.nih.gov/nuccore.” The NCBI is in the National Library of Medicine, Building 38A, Bethesda, MD 20894.
²American Type Culture Collection (ATCC) isolates can be accessed via the internet at the World Wide Web site “atcc.org.” The address for the ATCC is 10801 University Boulevard Manassas, VA 20110 USA. Culture Collection of Yeasts (CCY) isolates can be accessed via the internet at the world wide web site “ccy.sk.” The address for the CCY is Institute of Chemistry, Slovak Academy of Sciences, Dúbrayská cesta 9, 845 38 Bratislava, Slovakia. Pasteur Culture collection of Cyanobacteria (PCC) isolates can be accessed via the internet at the http address “cyanobacteria.web.pasteur.fr/.” The address for the PCC is Collections des Cyanobactéries, Institut Pasteur, 28, rue du Docteur Roux, 75724 PARIS Cedex 15, FRANCE.

Ratios of the cyanobacteria within the composition or coating can be varied. For example, equal parts of each cyanobacterium species can be provided in the composition or coating. Alternatively, a 3:1:1:1 mixture by mass of Nostoc sp., Aulosira sp., Anabaena sp., and Tolypothrix sp., can be used.

Ratios with a range of about 5:1, 4.5:1, 4:1, 3.5:1, 3:1, 2.5:1, 2:1, 1.5:1, 1.1:1, or 1:1 to about 1:1.1, 1:1.5, 1:2, 1:2; 1:2.5; 1:3, 1:3.5, 1:4, 1:4.5, or 1:5 of (i) an Aulosira sp., to a Tolypothrix sp.; or (ii) an Aulosira sp. to an Anabaena sp. can be used.

Ratios with a range of about 5:1, 4.5:1, 4:1, 3.5:1, 3:1, 2.5:1, 2.4:1, 2.3:1, 2.2:1, 2.1:1, or 2:1 to about 1.9:1, 1.8:1, 1.7:1, 1.6:1, 1.5:1, 1.4:1, 1.3:1, 1.2:1 or 1.1:1 of (i) an Aulosira sp., to a Tolypothrix sp.; or (ii) an Aulosira sp. to a Anabaena sp. can be used. For example, the Aulosira species and the Tolypothrix species can be in a ratio of 2.2:1 to 1.8:1.

Ratios ranging from about 3:1, 2.9:1, 2.8:1, 2.7:1, 2.6:1, or 2.5:1 to about 1.9:1, 1.8:1, 1.7:1, 1.6:1, 1.5:1, 1.4:1, 1.3:1, 1.2:1 or 1.1:1 of an Aulosira sp., to a Tolypothrix sp. can be used.

Ratios of about 1:1:1 of an Aulosira sp., an Anabaena sp., and a Tolypothrix sp. can be used.

The cyanobacteria described herein can be used to provide a total of about 0.0001 milligram to 5 milligram (dry weight) per seed. For example, a total of about 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 milligrams of the cyanobacterium in wet or dry weight per seed can be used. For example, a total of about 0.001 to about 0.05, 0.06, 0.07, 0.08, 0.09, or 0.1 milligram wet or dry weight of the cyanobacteria can be on the seed.

If a larger amount of coating per seed is desirable, from about 0.1, 0.2, or 0.3 to about 0.5, 0.6, 0.7, 0.8, 0.9, 1, 1.5, 2, 3, 4, or 5 milligrams per seed of the cyanobacterium in wet or dry weight can be used. For example, a total of 0.1 to about 0.9, 1, 1.5, 2, 3, 4, or 5 milligram wet or dry weight of the cyanobacteria can be on the seed.

The cyanobacterium can be provided at the equivalent of about 0.1, 0.2, 0.3, 0.4, or 0.5 kg to about 1, 1.1, 1.2, 1.3, 1.4, 1.5, 1.6, 1.7, 1.8, 1.9, 2, 2.5, 3, 3.5, 4, 4.5 or 5 kg wet weight per acre, or at the equivalent of about 12.5, 15, 20, 25, 30, 35, 40, 45, 50, 55, 60, 65, 70 or 75 grams to about 125, 150, 175, 200, 250, 300, 350, 400, 450 or 500 grams dry weight per acre of treated and sown seed.

For rice, the aforementioned wet weight or dry weight equivalents of cyanobacterium per acre of rice are obtained by sowing about 10,000, 20,000, 30,000, or 35,000 to about 45,000, 50,000, or 60,000 cyanobacterium treated rice seeds per acre. For wheat, the aforementioned wet weight or dry weight equivalents of cyanobacterium per acre of wheat are obtained by sowing about 0.8, 0.9, 1, 1.1, or 1.2 million to about 1.4, 1.5, 1.6, 1.7, or 1.8 million cyanobacterium treated wheat seeds per acre.

Agriculturally acceptable adjuvants used in the compositions or coatings can include, but are not limited to, one or more insecticides, fungicides, safeners, biological fertilizers, water retention compounds, nutrient retention compounds, biochar or a combination thereof.

Such adjuvants can be selected for an absence of bacteriocidal, bacteriostatic, or bacterio-inhibitory activities that would reduce the effectiveness of the cyanobacteria provided in the composition or coating.

The insecticide can comprise a carbamate, an organophosphate, a neonicotinoid, a pyrethroid, or a combination thereof.

A neonicotinoid insecticide can comprise thiamethoxam, imidacloprid, clothianidin, nitenpyram, nithiazine, thiacloprid, or a combination thereof.

The fungicide can include one or more of an azole, strobilurin, or a metalaxyl compound, or a combination thereof. Useful azole fungicides include, but are not limited to, difenoconazole, prothioconazole, and tebuconazole. Useful strobilurin fungicides include, but are not limited to, kresoxim-methyl, azoxystrobin, trifloxystrobin, fluoxastrobin, picoxystrobin, pyraclostrobin, dimoxystrobin, pyribencarb, metominostrobin, orysastrobin, enestrobin, pyraoxystrobin and pyrametostrobin. Useful metalaxyl fungicides include, but are not limited to, metalaxyl and mefenoxam.

Agriculturally acceptable excipients used in the compositions or coatings can include, but are not limited to, one or more bulking agents, binding agents, colorants, emulsifiers, oils, tackifiers, trace elements, and/or extending agents.

Useful bulking agents include, but are not limited to, peat, wood flour, calcium carbonate, lime, diatomaceous earth, forms of clay such as bentonite and kaolin, and combinations thereof.

Useful binding agents can be water soluble polymers and/or waxes. Binding agents that are used can include, but are not limited to, polyvinyl acetate, polyvinyl alcohol, polyvinyl pyrrolidone, polyurethane, methyl cellulose, carboxymethyl cellulose, hydroxypropyl cellulose, sodium alginate, polyurethane, polyacrylate, casein, gelatin, pullulan, polyacrylamide, polyethylene oxide, polystyrene, styrene acrylic copolymers, styrene butadiene polymers, poly(N-vinylacetamide), and combinations thereof. Waxes used as binders can include, but are not limited to, carnauba wax, paraffin wax, polyethylene wax, bees wax, and polypropylene wax.

Emulsifiers can be either ionic or non-ionic agents. Emulsifiers that can be used include, but are not limited to, lecithin, polysorbates, polyethylene glycols, derivatives thereof, and the like.

Oils that can be used include, but are not limited to, silicon, animal, plant, or mineral oils and mixtures thereof.

An exogenous tackifier can be added to the composition or coating. However, content of an exogenous tackifier can be reduced in comparison to exogenous tackifier content of a standard microbial seed inoculant.

Extenders are materials that provide for improvements in the viability of cyanobacteria on the seed either pre or post planting. Useful extenders include, but are not limited to, trehalose, sucrose, glycerol, sorbitol, and combinations thereof. Liquid seed treatment inoculums containing various microorganisms and extenders comprising one or more of trehalose, sucrose, glycerol or sorbitol at about 5% to about 50% by weight/volume and related methods where a partially desiccated liquid inoculant product for application to seeds is prepared are described in U.S. Pat. No. 8,020,343 and can be adapted to use with the cyanobacteria compositions or coatings provided herein. U.S. Pat. No. 8,020,343 is incorporated herein by reference in its entirety. Other liquid seed treatment compositions that can be adapted for use with the cyanobacteria compositions or coatings provide herein can comprise sucrose, sorbitol, or a combination thereof at about 5% to 60% weight/volume, mineral oil or silicon oil at about 0.15% to about 3% weight, and an emulsifying agent selected from the group consisting of lecithin and polysorbate and are described in U.S. Pat. No. 8,551,913. U.S. Pat. No. 8,551,913 is incorporated herein by reference in its entirety.

Exopolysaccharides (EPS) can be produced by the cyanobacteria in the composition or coating (“endogenous” EPS), by cyanobacteria not in the composition or coating, or by other bacteria (“exogenous” EPS). Exogenous EPS can also be used as agriculturally acceptable excipients in the compositions and coatings provided herein.

The exogenous EPS can be provided in the composition or coating by simply adding fermentation broths, filtrates, supernatants, purified fractions, partially purified fractions, and the like that are obtained from cyanobacterial or other bacterial cultures. Without seeking to be limited by theory, such EPS can improve water retention and/or desiccation tolerance of cyanobacteria in the compositions or coatings provided herein by slowing the desiccation process. It has been reported that bacterial EPS can help bacteria adapt to variable hydration conditions (Or et al. Advances in Water Resources, 30 (2007), pp. 1505-1527; Redmile-Gordon et al., Soil Biology & Biochemistry 72 (2014) 163e171). Exogenous exopolysaccharides obtained from sources other than cyanobacterial or other bacterial cultures that include, but are not limited to, fungal or yeast sources can be used either alone or in combination with the aforementioned cyanobacterial or bacterial cultures as agriculturally acceptable excipients.

Furthermore, a reduction in content of exogenous tackifier can be achieved from the endogenous EPS of the cyanobacteria of the composition or coating, or by using exogenous EPS as an agriculturally acceptable excipient in the composition or coating.

In the compositions and coatings described herein, water is not an excipient or adjuvant. To the extent that water content of a seed increases after it is coated and lyophilized or dried, the increase in water content is generally observed within the cyanobacteria itself.

The composition or coating can contain at least one element or a salt thereof comprising iron, boron, manganese, zinc, molybdenum, copper, cobalt, or a combination thereof.

The treated seeds can be used to improve: (i) biomass, grain yield, nutrient uptake and/or nitrogen uptake in a monocot plant including, but not limited to a rice, a corn, a sorghum, a turfgrass, a wheat, a biofuel crop, a forage crop, or a millet plant; (ii) plant growth in a monocot plant, including, but not limited to, improved growth in comparison to a monocot plant obtained from an untreated control seed where the plants are grown under conditions where nitrogen fertilizer is not used, a suboptimal amount of nitrogen fertilizer is used, or where less than 40, 50, 60, 70, 80, 90. 100, 150 or 200 lbs of nitrogen fertilizer per acre is used; or (iii) biomass, grain yield, nutrient uptake and/or nitrogen uptake in a rice plant obtained from the rice seed including, but not limited to, improvements in comparison to a rice plant obtained from an untreated control rice seed.

Grain yield resulting from the treated monocot plant seed other than a rice seed can be at least 2.0% greater than grain yield resulting from the same monocot plant seed that is not treated with the coating when grown under greenhouse conditions. For example, grain yield resulting from the treated monocot plant seed can be at least 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater than grain yield resulting from the same monocot plant seed that is not treated with the coating when grown under greenhouse conditions as defined below. For example, grain yield can be increased by about 2-8% in corn or wheat.

Grain yield resulting from the treated rice seed can be at least 10.0% greater than grain yield resulting from the same rice seed that is not treated with the coating when grown under greenhouse conditions. For example, grain yield resulting from the treated rice seed can be at least 11.0, 12.0, 13.0, 14.0, 15.0, 16.0, 17.0, 18.0, 19.0, 20.0, 21.0, 22.0, 23.0, 24.0, or 25.0% greater than grain yield resulting from the same rice seed that is not treated with the coating when grown under greenhouse conditions as defined below.

Grain yields increases of as much as 35% can be obtained.

Nitrogen uptake resulting from the treated monocot plant seed can be at least 5% greater than nitrogen uptake resulting from the same monocot plant seed that is not treated with the coating when grown under the greenhouse conditions. For example, nitrogen uptake resulting from the treated monocot plant seed can be at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater than nitrogen uptake resulting from the same monocot plant seed that is not treated with the coating when grown under the greenhouse conditions.

Nitrogen uptake increases of as much as 80% can be obtained.

Nutrient uptake resulting from the treated monocot plant seed can be at least 2% greater than nutrient uptake resulting from the same monocot plant seed that is not treated with the coating when grown under the greenhouse conditions. For example, nutrient uptake resulting from the treated monocot plant seed can be at least 3, 4, 5, 6, 7, 8, 9, or 10% greater than nutrient uptake resulting from the same monocot plant seed that is not treated with the coating when grown under the greenhouse conditions.

Biomass resulting from the treated monocot plant seed can be at least 2% greater than biomass resulting from the same monocot plant seed that is not treated with the coating when grown under the greenhouse conditions. For example, biomass resulting from the treated monocot plant seed can be at least 3, 4, 5, 6, 7, 8, 9, 10, 11, 12, 13, 14, 15, 16, 17, 18, 19, 20, 21, 22, 23, 24, or 25% greater than biomass resulting from the same monocot plant seed that is not treated with the coating when grown under the greenhouse conditions.

Biomass increases of as much as 35% can be obtained.

Lateral root growth, lateral root hair growth, vertical root growth, or root branching resulting from the treated monocot plant seed can exhibit increased branching as compared to lateral root growth, lateral root hair growth, vertical root growth, or root branching resulting from the same monocot plant seed that is not treated with the coating when grown under the greenhouse conditions.

Also provided is a method of obtaining a monocot plant seed that provides for improved growth in a monocot plant obtained from the seed comprising applying a composition comprising the cyanobacteria to the at least a portion of an outer surface of the seed and lyophilizing or drying the composition to form the solid coating and obtain the treated monocot plant seed. The composition can be applied as a liquid or a slurry.

Also provided is a method of obtaining a plurality of monocot crop plants with improved grain yield, nitrogen uptake, nutrient uptake, biomass, lateral root growth, lateral root hair growth, vertical root growth, or root branching, the method comprising: distributing the treated monocot plant seed on a plot of land and cultivating plants grown from the seed on the plot.

The methods can further comprise harvesting the cultivated plants or grain therefrom.

Also provided are methods of obtaining a monocot plant seed that provides for improved growth in a monocot plant obtained from the seed, the method comprising: applying a composition comprising: (i) at least one cyanobacterium species; and (ii) an agriculturally acceptable adjuvant, an agriculturally acceptable excipient, or combination thereof to at least one surface of the seed to obtain a seed that is at least partially coated with the composition, wherein growth of a monocot plant obtained from the treated monocot plant seed is increased in comparison to growth in a monocot plant obtained from an untreated control monocot plant seed.

Also provided are methods of obtaining a corn seed that provides for improved lateral root growth or lateral root hair growth in a seedling obtained from the corn seed, the method comprising: applying a composition comprising: (i) at least one cyanobacterium species; and (ii) an agriculturally acceptable adjuvant, an agriculturally acceptable excipient, or combination thereof to at least one surface of the seed to obtain a seed that is at least partially coated with the composition, wherein lateral root growth or lateral root hair growth in a seedling obtained from the treated corn seed is increased in comparison to lateral root growth or lateral root hair growth in a seedling obtained from an untreated control corn seed.

Also provided are methods of obtaining a rice seed that provides for improved biomass, grain yield, nutrient uptake and/or nitrogen uptake in a rice plant obtained from the rice seed, the method comprising: applying a composition comprising: (i) at least one cyanobacterium species; and (ii) an agriculturally acceptable adjuvant, an agriculturally acceptable excipient, or combination thereof to at least one surface of the seed to obtain a seed that is at least partially coated with the composition, wherein biomass, grain yield, nutrient uptake and/or nitrogen uptake in a rice plant obtained from the treated rice seed is increased in comparison to biomass, grain yield, nutrient uptake and/or nitrogen uptake in a rice plant obtained from an untreated control rice seed.

In the methods described herein, the composition can be applied as a liquid or slurry.

The composition can be lyophilized or dried upon the surface of the seed.

Also provided is a method of improving grain yield, nitrogen uptake, nutrient uptake, biomass, lateral root growth, lateral root hair growth, vertical root growth, or root branching in a monocot plant, the method comprising:

exposing the monocot plant seed to an effective amount of a composition comprising cyanobacteria comprised of an Aulosira species and a Tolypothrix species, wherein either:

(1) the composition is free of an agriculturally acceptable adjuvant and/or an agriculturally acceptable excipient; or

(2) the composition further comprises the adjuvant and/or the excipient in an amount less than 0.4 kg/hectare; or

(3) the cyanobacteria consists essentially of the Aulosira species and the Tolypothrix species; or

(4) the composition consists essentially of the Aulosira species and the Tolypothrix species; or

(5) the composition comprises the Aulosira species and the Tolypothrix species in a weight ratio of about 5:1 to about 1:5; and allowing the monocot plant seed to germinate;

wherein either:

(A) grain yield resulting from the treated monocot plant seed other than rice is at least 2.0% greater than grain yield resulting from the same monocot plant seed that is not exposed to the composition when grown under greenhouse conditions; or

(B) grain yield resulting from the treated rice seed is at least 10.0% greater than grain yield resulting from the same rice seed that is not exposed to the composition when grown under greenhouse conditions; or

(C) nitrogen uptake resulting from the treated monocot plant seed is at least 5% greater than nitrogen uptake resulting from the same monocot plant seed that is not exposed to the composition when grown under the greenhouse conditions; or

(D) biomass resulting from the treated monocot plant seed is at least 2% greater than biomass resulting from the same monocot plant seed that is not exposed to the composition when grown under the greenhouse conditions; or

(E) lateral root growth, lateral root hair growth, vertical root growth, or root branching resulting from the treated monocot plant seed exhibits increased branching as compared to lateral root growth, lateral root hair growth, vertical root growth, or root branching resulting from the same monocot plant seed that is not exposed to the composition when grown under the greenhouse conditions; or

(F) nutrient uptake resulting from the treated monocot plant seed is at least 2% greater than nutrient uptake resulting from the same monocot plant seed that is not exposed to the composition when grown under the greenhouse conditions;

wherein the greenhouse conditions are as follows:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) for rice, permanent flood is established at 4-leaf growth stage;

(c) temperature maintained at 25-28° C., 50-70% relative humidity and natural light supplemented with fluorescent light at 35 μmol m⁻² s⁻² photosynthetically active radiation to provide a 14 h photoperiod; and

(d) plant grown to 50% heading then entire above-ground biomass is collected and dried, and total weight and nitrogen concentration can be measured.

Increases in grain yield, nitrogen uptake, nutrient uptake, biomass, lateral root growth, lateral root hair growth, vertical root growth, or root branching can be as described above. For example, grain yield resulting from the treated monocot plant seed other than rice can be at least 2.0% greater (e.g., at least 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater) than grain yield resulting from the same monocot plant seed that is not exposed to the composition when grown under greenhouse conditions. Grain yield resulting from the treated rice seed can be at least 10.0% greater (e.g., at least 11.0, 12.0, 13.0, 14.0, 15.0, 16.0, 17.0, 18.0, 19.0, 20.0, 21.0, 22.0, 23.0, 24.0, or 25.0% greater) than grain yield resulting from the same rice seed that is not exposed to the composition when grown under greenhouse conditions. Nitrogen uptake resulting from the treated monocot plant seed can be at least 5% greater (e.g., at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater) than nitrogen uptake resulting from the same monocot plant seed that is not exposed to the composition when grown under the greenhouse conditions. Nutrient uptake resulting from the treated monocot plant seed can be at least 2% greater (e.g., at least 3, 4, 5, 6, 7, 8, 9, or 10% greater) than nutrient uptake resulting from the same monocot plant seed that is not treated with the coating when grown under the greenhouse conditions. Biomass resulting from the treated monocot plant seed can be at least 2% greater (e.g., at least 3, 4, 5, 6, 7, 8, 9, 10, 11, 12, 13, 14, 15, 16, 17, 18, 19, 20, 21, 22, 23, 24, or 25% greater) than biomass resulting from the same monocot plant seed that is not exposed to the composition when grown under the greenhouse conditions. Lateral root growth, lateral root hair growth, vertical root growth, or root branching resulting from the treated monocot plant seed can exhibit increased branching as compared to lateral root growth, lateral root hair growth, vertical root growth, or root branching resulting from the same monocot plant seed that is not exposed to the composition when grown under the greenhouse conditions.

Also provided are methods of improving lateral root growth or lateral root hair growth in a corn seedling, the method comprising: exposing a corn seed to an effective amount of a composition comprising: (i) at least one cyanobacterium species; and (ii) an agriculturally acceptable adjuvant or an agriculturally acceptable excipient and allowing the seed to germinate, wherein lateral root growth or lateral root hair growth in a seedling obtained from the corn seed exposed to the composition is increased in comparison to lateral root growth or lateral root hair growth a seedling obtained from a control corn seed that was not exposed to the composition.

In the methods described herein, the exposing step can comprise applying the composition to the seed by either in furrow application or by a soil drench application.

In the methods described herein, the exposing step can comprise applying the composition to a surface of the seed to obtain a seed that is at least partially coated with the composition.

Also provided are methods of improving plant growth in a monocot plant, the method comprising: exposing a seed to an effective amount of a composition comprising: (i) at least one cyanobacterium species; and (ii) an agriculturally acceptable adjuvant, an agriculturally acceptable excipient, or combination thereof and allowing the seed to germinate, wherein growth of a monocot plant obtained from the seed exposed to the composition is increased in comparison to growth of a monocot plant obtained from a control monocot plant seed that was not exposed to the composition.

In the methods described herein, the seed can be exposed to a composition or the composition can be applied to the seed, the composition comprising: (i) an Aulosira species and a Tolypothrix species at a ratio of 3:1 to 1.5:1, respectively; and (ii) an agriculturally acceptable adjuvant, an agriculturally acceptable excipient, or a combination thereof.

Also provided are methods of improving biomass, grain yield, nutrient uptake and/or nitrogen uptake in a monocot plant, the method comprising exposing a monocot seed to an effective amount of a composition comprising: (i) at least one cyanobacterium species; and (ii) an agriculturally acceptable adjuvant, an agriculturally acceptable excipient, or combination thereof and allowing the seed to germinate, wherein biomass, grain yield, nutrient uptake and/or nitrogen uptake in a monocot plant obtained from the monocot seed exposed to the composition is increased in comparison to biomass, grain yield, nutrient uptake and/or nitrogen uptake in a monocot plant obtained from a control monocot seed that was not exposed to the composition. For example, the monocot plant can be a rice plant.

Any method of measuring the growth characteristic can be employed. Typically, the biomass, grain yield, and the like can be assessed by determining the mass of plant material obtained, the mass of seeds obtained, or the number of seeds and a per plant or per unit area (e.g., per acre or hectare) basis. Lateral root growth, lateral root hair growth, vertical root growth, or root branching can be measured by visual inspection to determine length and/or width or by imaging to quantify the length, width, density, volume or branching of roots and root systems.

Seeds treated with compositions or coatings provided herein can be used to improve monocot plant growth under nitrogen limiting conditions and/or to improve nitrogen uptake by monocot plants. Plant growth can improve under conditions where nitrogen is limiting. When a suboptimal amount of nitrogen fertilizer is used, a minimum of 0.1 lb/acre can be used.

The plant growth medium in which the plant seed is grown need not be supplemented with nitrogen if desired.

In the methods described herein, it may be desirable not to use nitrogen fertilizer, or to use a suboptimal amount of nitrogen fertilizer. For example, less than 5, 10, 15, 20, 25, 30, 35, 40, 45, 50, 60, 70, 80, 90, 100, 110, 120, 130, 140, 150, 160, 170, 180, 190 or 200 lbs/acre of nitrogen fertilizer can be used in the methods.

Where nitrogen fertilizer is not used or where a suboptimal amount of nitrogen fertilizer is used, compositions or coatings comprising: (i) an Aulosira sp., and a Tolypothrix sp.; (ii) an Aulosira sp.; (iii) an Aulosira sp. and an Anabaena sp.; (iv) an Aulosira sp., an Anabaena sp., and a Tolypothrix sp.; or (v) a Nostoc sp. can be used on rice seed.

Where nitrogen fertilizer is not used or where a suboptimal amount of nitrogen fertilizer is used, compositions or coatings comprising (i) Aulosira bohemensis and Tolypothrix distorta; (ii) Aulosira bohemensis; (iii) Aulosira bohemensis and Anabaena cylindrica; (iv) an Aulosira bohemensis, an Anabaena cylindrica sp., and a Tolypothrix distorta sp.; or (v) a Nostoc commune sp. can be used on corn seed.

Where nitrogen fertilizer is not used or where a suboptimal amount of nitrogen fertilizer is used, compositions or coatings comprising (i) Aulosira bohemensis and Tolypothrix distorta; (ii) Aulosira bohemensis; (iii) Aulosira bohemensis and Anabaena cylindrica; (iv) an Aulosira bohemensis, an Anabaena cylindrica sp., and a Tolypothrix distorta sp.; or (v) a Nostoc commune sp. can be used on wheat seed. The amount of nitrogen fertilizer used is usually less than an equivalent of 110 lbs/acre of nitrogen.

Improvements in monocot plant growth or monocot plant yield obtained by seed treatments with cyanobacterial compositions or coatings provided herein under any of the aforementioned nitrogen limiting conditions can be at least about 3, 4, 9, 10, 15, or 20% in comparison to an untreated control monocot plant or can be about 3, 4, 5, or 10% to about 15, 18, 20, 30, or 40% in comparison to an untreated control monocot plant.

Improvements in monocot plant growth or monocot plant yield under any of the aforementioned nitrogen limiting conditions with a cyanobacterial seed treatment provided herein can be at least about 10% in comparison to an untreated control monocot plant or can be about 10, 15, 18, or 25% in comparison to a monocot plant subjected to a topical treatment of the cyanobacteria.

Nitrogen uptake by plants obtained from seeds treated with the cyanobacterial compositions or coatings provided herein can be improved under nitrogen limiting conditions and under conditions where nitrogen is not limiting. Nitrogen uptake can be determined by a variety of methods that include, but are not limited to, isotopic and non-isotopic methods that have been described (Sandrock et a. Hort. Sci. 40(3):665, 2005; Norman et al. Soil Sci. Soc. Am. J. 56:1521-1527. 1992).

Improvements in nitrogen uptake by monocot plants obtained by seed treatments with cyanobacterial compositions or coatings provided herein under any of the aforementioned nitrogen limiting conditions can be at least about 3, 4, 5, 10, 20, 40, 50, or 60% in comparison to an untreated control monocot plant or can be about 3, 4, 5, or 10%, to about 15, 18, 20, 40, 60, or 70% in comparison to an untreated control monocot plant.

Improvements in nitrogen uptake under any of the aforementioned nitrogen limiting conditions with a cyanobacterial seed treatment provided herein can be at least about 3, 4, 9, or 10% in comparison to an untreated control monocot plant or can be about 3, 4, or 5% to about 10, 15, 20, 30, or 40% in comparison to a monocot plant subjected to a topical treatment of the cyanobacteria.

In the methods and seed treatments described herein, the seed can be dormant before, during, and/or after treatment.

The monocot plant or monocot plant seed can comprise rice, corn, sorghum, turfgrass, wheat, a biofuel crop, a forage crop, or millet. The turfgrass plant or seed can comprise bentgrass, bermudagrass, bluegrass, buffalograss, fescue, or ryegrass. The forage crop plant or seed can be hay, ryegrass, or oat. The biofuel crop can be a Miscanthus or switchgrass.

The seed can be a rice seed and wherein biomass, grain yield, nutrient uptake and/or nitrogen uptake of a plant obtained from the treated rice seed is increased in comparison to biomass, grain yield, nutrient uptake and/or nitrogen uptake of a plant obtained from an untreated control rice seed.

The seed can be a corn seed wherein lateral root growth or lateral root hair growth in a seedling obtained from the treated corn seed is increased in comparison to lateral root growth or lateral root hair growth in a seedling obtained from an untreated control corn seed.

Seed coating equipment and associated techniques used to coat the seeds include, but are not limited to, drum coaters, fluidized beds, rotary coaters, side vended pan, tumble mixers, and spouted beds. Conventional seed coating methods for use herein are well known in the art.

Other Treated Particles

The treated monocot plant seeds as described herein can provide a natural, non-synthetic, organic and biodegradable means for binding an agriculturally acceptable adjuvant and/or an agriculturally acceptable excipient to a seed. Under high humidity environments, a conventional coating may not adhere well to a seed and exhibit bridging and agglomeration, but the coating of the treated monocot plant seeds described herein adheres well to the seed and tolerates high humidity conditions.

For these reasons, the coating can be used to bind agriculturally acceptable adjuvants and/or an agriculturally acceptable excipients to a particle other than a seed, such as a fertilizer granule, biological fertilizer, water or nutrient retention device, or a biochar particle.

Also provided is a treated particle comprising a solid coating on at least a portion of an outer surface of the particle, the coating comprising a cyanobacterium comprised of an Aulosira species, and the particle comprising a fertilizer granule, biological fertilizer, water or nutrient retention device, or a biochar particle.

The fertilizer granule can be any fertilizer particle, including those containing one or more of: nitrogen, phosphate, potassium, other minerals, and/or other plant nutrients. Such fertilizer granules are well known in the art and widely commercially available.

A biological fertilizer can be any other seed coating or agricultural treatment that increases agricultural productivity by making nutrients better available to plants or reduces deleterious interactions.

A water or nutrient retention device can be any coating or granule that is designed to improve agricultural productivity by increasing availability of critical growth components, including but not limited to water, nitrogen, potassium, phosphorus and other trace fertilizers.

The biochar particle can be any charcoal such as charcoal used as a soil amendment. Biochar is commercially available from various sources.

Biochar can hold carbon, increase soil biodiversity, and help soils retain nutrients, agrochemicals, and water. Biochar can be naturally found in soils, for example, as a result of vegetation fires. Biochar allows more nutrients to stay in the soil instead of leaching into groundwater and causing pollution. The carbon in biochar resists degradation. Biochar is produced through pyrolysis or gasification processes that heat biomass in the absence or under reduced amounts of oxygen.

The cyanobacterium can be as described above for the treated monocot plant seeds. For example, the cyanobacterium can further comprise a Tolypothrix species.

The cyanobacterium can be present in an amount of about 1-45 wt. % of the coating.

The coating can be as described above for the treated monocot plant seeds, including the adjuvants and excipients as described above.

Preferably, the adjuvant and/or excipient is different than the particle. For example, when the particle comprises a biochar particle, the agriculturally acceptable adjuvant comprises an insecticide, a fungicide, a safener, biological fertilizers, water retention compounds, nutrient retention compounds, or a combination thereof.

The adjuvant and/or excipient can be present in an amount of about 55-99 wt. % of the coating.

The coating for the particle can be free of either the adjuvant or the excipient if desired.

The coating can be in an amount from about 0.0001 to about 5 mg (dry weight) per particle, or from about 0.0005 to about 4 mg (dry weight) per particle, or from about 0.001 to about 3 mg (dry weight) per particle, or from about 0.0001, 0.0005, 0.001, 0.005, 0.01, or 0.05 to 0.5, 0.6, 0.7, 0.8, 0.9, 1, 1.5, 2, 2.5, 3, 3.5, 4 or 4.5 mg (dry weight) per particle.

The adjuvant and/or excipient in the coating can be in an amount from about 0.0001 to about 500 mg (dry weight) per particle, or from about 0.0005 to about 400 mg (dry weight) per particle, from about 0.001 to about 300 mg (dry weight) per particle, or from about 0.0001, 0.0005, 0.001, 0.005, 0.01, 0.05, 0.1, 0.2, 0.3, or 0.4 to 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mg (dry weight) per particle.

The treated particle can be non-naturally occurring. For example, the Aulosira species and/or the Tolypothrix species may not be associated with the particle in nature or (ii) the coating may comprise an exogenous exopolysaccharide not associated with the particle in nature.

Also provided is a method of binding an agriculturally acceptable adjuvant and/or an agriculturally acceptable excipient to a particle, the method comprising: applying a composition comprising the cyanobacteria and the adjuvant and/or excipient to the at least a portion of an outer surface of the particle and lyophilizing or drying the composition to form the solid coating and obtain the treated particle as described herein.

Also provided is a method of obtaining a plurality of plants comprising: distributing the treated particles on a plot of land; planting the plot with seeds or plants; and cultivating plants grown on the plot. The method can further comprise harvesting the cultivated plants.

Also provided is a method of obtaining a treated particle comprising: applying a composition comprising the cyanobacteria to the at least a portion of an outer surface of the particle and lyophilizing or drying the composition to form the solid coating and obtain the treated particle.

The composition can be applied as a liquid or slurry.

### Definitions

Any ranges described herein in the form of “A, B, or C to X, Y, or Z” means that every possible combination of such ranges are disclosed herein (i.e., A to X, A to Y, A to Z, B to X, B to Y, B to Z, C to X, C to Y, and C to Z). For example, a range of “about 3%, 4%, or 5% to about 10%, 15%, 20%, 30%, or 40%” means that the ranges “about 3% to about 10%, about 3% to about 15%, about 3% to about 20%, about 3% to about 30%, about 3% to about 40%, about 4% to about 10%, about 4% to about 15%, about 4% to about 20%, about 4% to about 30%, about 4% to about 40%, about 5% to about 10%, about 5% to about 15%, about 5% to about 20%, about 5% to about 30%, and about 5% to about 40%” are described. As another example, a range of ratios of “about 3:1, 2.5:1, or 2.2:1 to about 1.8:1, 1.7:1, or 1.5:1” means that the ranges “about 3:1 to about 1.8:1, about 3:1 to about 1.7:1, about 3:1 to about 1.5:1, about 2.5:1 to about 1.8:1, about 2.5:1 to about 1.7:1, about 2.5:1 to about 1.5:1, about 2.2:1 to about 1.8:1, about 2.2:1 to about 1.7:1, about 2.2:1 to about 1.5:1” are described.

The term “consisting essentially of” as used herein means that the composition, coating or cyanobacteria contains the specified ingredients, as well as additional unspecified ingredients, provided that the unspecified ingredients do not materially affect the basic and novel characteristics of the composition, coating, or cyanobacteria. These characteristics include increased grain yield, nitrogen uptake, nutrient uptake, biomass, lateral root growth, lateral root hair growth, vertical root growth, or root branching as compared to seeds untreated with the composition, coating or cyanobacterium under greenhouse conditions as specified herein. The composition, coating or cyanobacterium excludes, for example, an amount of any cyanobacterium species (such as Nostoc species) that would decrease grain yield, nitrogen uptake, nutrient uptake, biomass, lateral root growth, lateral root hair growth, vertical root growth, and/or root branching as compared to the same composition, coating or cyanobacteria that does not include the cyanobacterium species when grown under the specified greenhouse conditions.

As used herein in reference to a cyanobacterium preparation, “dry weight” refers to the weight of cyanobacterium that have been both separated from liquid and dried. Such drying can be effected by methods including, but not limited to, evaporation, lyophilization, or combinations thereof.

“Endogenous EPS” is EPS produced by any cyanobacterium within the composition or coating.

“Exogenous EPS” is EPS produced by any cyanobacterium not in the composition or coating, or produced by bacteria other than a cyanobacterium.

When “greenhouse conditions” are specified for comparative results herein, the conditions are as follows when using treated seeds:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) for rice, permanent flood is established at 4-leaf growth stage;
(c) temperature maintained at 25-28° C., 50-70% relative humidity and natural light supplemented with fluorescent light at 35 μmol m⁻² s⁻² photosynthetically active radiation to provide a 14 h photoperiod; and
(d) plant grown to 50% heading then entire above-ground biomass is collected and dried, and total weight and nitrogen concentration can be measured.

As used herein, the term “lateral root” refers to a root that originates from a primary or nodal root.

As used herein, the term “lateral root hair” refers to a root hair that originates from a lateral root.

As used herein, the terms “nitrogen limiting conditions” or “a suboptimal amount of nitrogen fertilizer is used” refer to conditions where plant growth or yield can be increased upon supplementing the plant growth medium (e.g., soil, synthetic rooting mixes, hydroponic liquids, flooded fields, and the like) with exogenous nitrogen or by improving the availability of nitrogen already present in the plant growth medium.

A “nutrient” is defined as a mineral or inorganic compound that is absorbed from soil or other growth medium that is essential for plant growth or metabolism. Nutrients include, but are not limited to, nitrogen, phosphorus, potassium, calcium, sulfur, magnesium, boron, chlorine, manganese, iron, zinc, copper, molybdenum, and nickel.

“Root branching” is defined herein as the process in which lateral roots grow out from the taproot and other lateral roots for the purpose of providing plant stability and nutrient uptake.

“Vertical root growth” is defined herein as the elongation of the root, taproot, and seminal roots in a direction perpendicular to the soil surface.

As used herein in reference to a cyanobacterium preparation, “wet weight” refers to the weight of cyanobacterium that have been separated from liquid but not dried. Such separation can be effected by methods including, but not limited to, settling/decanting, filtration, centrifugation, or combinations thereof.

Unless otherwise defined, all technical and scientific terms used herein have the same meaning as commonly understood by one of ordinary skill in the art to which this disclosure pertains. In the case of inconsistencies between the present disclosure and the issued patents, applications, and references that are cited herein, the present disclosure will prevail.

Having described the invention in detail, it will be apparent that modifications and variations are possible without departing from the scope of the invention defined in the appended claims.

### EXAMPLES

The following non-limiting examples are provided to further illustrate the present invention.

### Example 1. Microbial Biomass Production

Nostoc commune UTEX B 1621, Anabaena cylindrica UTEX B 1611, Aulosira bohemensis UTEX B 2947, and Tolypothrix distorta UTEX 424 were maintained under constant 105 microMol photons*m⁻²*sec⁻¹ shaded cool white fluorescent light on sterile BG11 plates. BG11 media has been described (Rippka Archiv für Mikrobiologie 87, Issue 1, pp 93-98, 1972) and is commercially available (Sigma-Aldrich, St. Louis, Mo.). Plates were re-streaked onto fresh plates as colonies appeared using a microbiological loop under sterile conditions. For amplification, biomass from plates was transferred into several autoclaved 250 ml Erlenmeyer flasks containing 100 ml sterile BG11 by swishing a sterile microbiological loop containing microbial biomass (obtained from the plates) in the liquid media. The Erlenmeyer flasks were stoppered with autoclaved foam stoppers.

The 250 ml Erlenmeyer flasks containing monocultures were shaken at 120 rpm on an orbital shaker in constant 40 microMol photons*m⁻²*sec⁻¹ cool white fluorescent light at 22° C. until cultures reached translucent green cell density. The entirety of four 250 ml Erlenmeyer flasks with the same species of cyanobacteria, containing 100 ml of BG11 each, was then transferred to autoclaved tubular 4 L flasks containing 3.5 L sterile BG11. The 4 L bubble flasks were mixed by aeration through an autoclaved bubble tube connected to an aquarium pump. A filter made of autoclaved cotton fibers was placed in the tubing line to maintain sterility. An autoclaved foam stopper with a hole for the air-in port was used to seal the 4 L flasks. The culture was supplied with constant 45 microMol photons*m⁻²*sec⁻¹ cool white fluorescent light at 22° C.

Upon reaching translucent green cell density, 2 L culture from the 4 L bubble flasks was transferred to a 20 L Pyrex carboy containing 14 L sterile BG11 (for a total culture volume of 16 L). The 4 L bubble flask was replenished with 2 L sterile BG11 to allow for additional 20 L inoculations as necessary. Monocultures growing in 20 L carboys were diluted by half with fresh sterile BG11 as necessary to maintain dense, translucent green cultures. As necessary, upon dilution with fresh BG11, excess culture was transferred to plastic 20 L carboys that were washed with antibacterial soap and sterilized with ethanol. These cultures were also supplied with constant 45 microMol photons*m⁻²*sec⁻¹ at 22° C.

Harvesting of cyanobacterial monocultures began by settling monocultures growing in 20 L carboys for 2-4 hours (total settling time depended on time it takes individual carboys to settle). After settling, clear supernatant was siphoned off using autoclaved plastic tubing. A total of 1-6 L of each monoculture remained per 20 L carboy after settling. Settled cultures were then pulse blended in an Oster blender for less ˜5 seconds. Cultures were then centrifuged at 6,000×g for 10 minutes in either a 6×50 ml benchtop centrifuge or a 6×1 L floor centrifuge. Centrifuge size was chosen based upon total biomass required for subsequent plant treatments. Each vessel was decanted after centrifugation, and biomass pellets were combined into a sterilized beaker. Total wet weight biomass was determined by measuring the weight of the beaker before and after the addition of biomass. Biomass cyanobacterial mixtures were prepared by mixing biomass paste of each monoculture by weight with the other members of the cyanobacterial mixture to give the prescribed ratio. Biomass paste was then diluted to a working volume with sterile Millipore water, giving a known working biomass concentration.

### Example 2. Corn Seed Coating

Two cyanobacterial application rates were used to coat corn seeds: 1 and 4 mg per seed. A control was also created with no cyanobacteria applied. Cyanobacterial biomass was prepared by harvesting excess liquid Nostoc cyanobacterial monoculture, centrifuging, and removing the supernatant. The culture was centrifuged in twelve 50 ml Falcon tubes for 10 min at 6000×g. The biomass pellets were then diluted with a small amount of Nanopure™ water (Thermo Fischer Scientific, Inc. USA), harvested and combined into one tube. The cyanobacterial culture was then homogenized by blending on a pulse setting in a kitchen blender for less than five seconds. As little blending as possible was used to prevent unnecessary cell damage. After homogenization, the biomass was spun for 10 min at 6000×g to remove any remaining liquid. Cyanobacterial biomass was added to individual 15 ml falcon tubes as a paste. Biomass was calculated by subtracting the weight of the tube and paste from the weight of the tube only (determined at beginning of experiment). The 0, 1, and 4 mg per seed application rates were diluted with Nanopure™ water to a total volume of 12 ml before coating. Six replicates of each application rate were prepared. Four were used in seed coating and two served as backups in case there were unforeseen issues with coating.

Two corn varieties, DKC 59-43 and DKC 63-43 (DeKalb varieties from Monsanto, St. Louis, Mo. USA) were coated with 1 and 4 mg per seed of Nostoc commune cyanobacteria (prepared as above) using Hege™ 11 liquid seed treater. All seeds received a mixture of 0.064 mg per seed of the fungicide Maxim™ and 0.046 mg per seed of the binder Flo Rite™ 1197 with inert colorant Color Coat Red. Each cyanobacterial treatment was performed in duplicate, with one subset receiving Cruiser™ seed-applied insecticide and one subset receiving no insecticide. The chemicals were added to the Falcon™ tubes (Thermo Fisher Scientific Inc, Waltham, Mass., USA) containing the cyanobacteria (previous paragraph) and diluted to a volume of 12 ml with Nanopure™ water before coating. Coating was performed in 3 applications of 4 ml per application. Between coatings, seeds were allowed to dry on a cookie sheet at room temperature for four hours. Drying between applications was done to prevent early germination.

TABLE 4
Seed coating subgroups for corn trials

| | | Nostoc | |
|---|---|---|---|
| | | Amount | |
| Group | Seed | (mg) | Cruiser ™ |
| 1 | DKC 59-43 | 0 | + |
| 2 | DKC 59-43 | 0 | − |
| 3 | DKC 59-43 | 1 | + |
| 4 | DKC 59-43 | 1 | − |
| 5 | DKC 59-43 | 4 | + |
| 6 | DKC 59-43 | 4 | − |
| 7 | DKC 63-43 | 0 | + |
| 8 | DKC 63-43 | 0 | − |
| 9 | DKC 63-43 | 1 | + |
| 10 | DKC 63-43 | 1 | − |
| 11 | DKC 63-43 | 4 | + |
| 12 | DKC 63-43 | 4 | − |

In each subset of corn seeds, two hundred seeds were coated in batch with a given cyanobacterial amount (0, 1, and 4 mg per seed), laid out onto a single layer onto cookie sheets, and allowed to dry at room temperature in a well-ventilated area overnight between the second and third applications. In total 2,400 seeds were coated, 800 of which were controls.

DKC 59-43 and DKC 63-43 were successfully coated with Nostoc commune UTEX B 1621.

### Example 3. Germination and Root Hair Formation

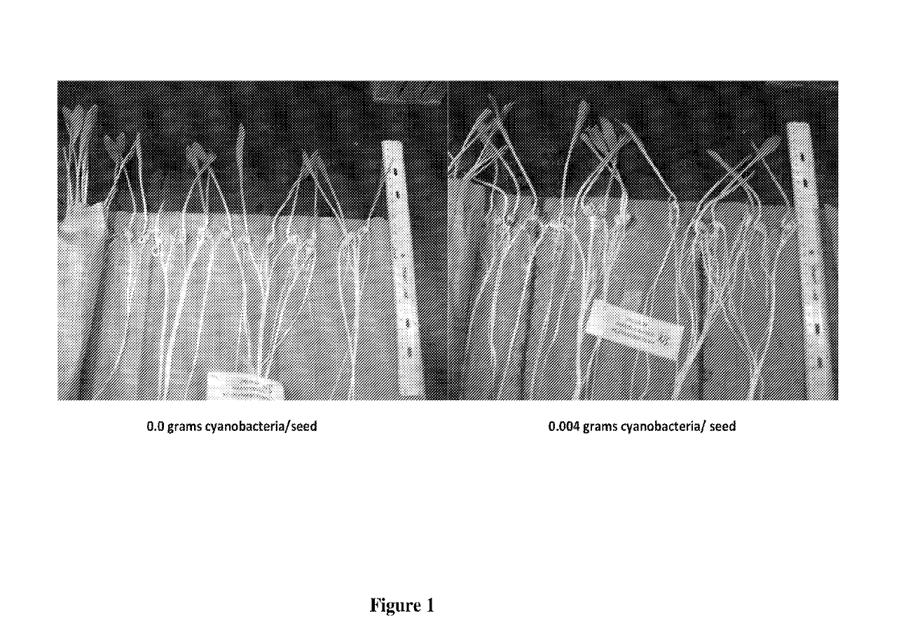

Seeds from Table 4 of Example 2 were germinated and images of the roots were captured daily for one week. There was found to be a dramatic increase in the formation of lateral root hairs in seedlings obtained from seeds treated with 4 mg of cyanobacteria (FIG. 1).

### Example 4. Seed Treatment of High Performance Corn Varieties

Cyanobacterium Mixture A, Mixture B, and Mixture C were prepared essentially as described in Example 1. Mixture A comprises: Nostoc commune UTEX B1621 alone. Mixture B was a 3:1:1:1 mixture of Nostoc commune UTEX B 1621, Anabaena cylindrica UTEX B 1611, Aulosira bohemensis UTEX B 2947, and Tolypothrix distorta UTEX 424. Mixture C was a 1:1:1 mixture of Aulosira bohemensis UTEX B 2947, Anabaena cylindrica UTEX B 1611, and Tolypothrix distorta UTEX 424. About 4 mg of each cyanobacterial mixture was applied per seed. A control was also created with no cyanobacteria applied. Cyanobacterial biomass was prepared by harvesting excess liquid Nostoc commune UTEX B 1621, Aulosira bohemensis UTEX B 2947, Anabaena cylindrica UTEX B 1611, and Tolypothrix distorta UTEX 424 cyanobacterial monocultures, centrifuging, and removing the supernatant. The culture was centrifuged in twelve 50 ml Falcon tubes for 10 min at 6000×g. The biomass pellets were then diluted with a small amount of Nanopure™ water, harvested and combined into one tube. The cyanobacterial culture was then homogenized by blending on a pulse setting in a kitchen blender for less than five seconds. As little blending as possible was used to prevent unnecessary cell damage. After homogenization, the biomass was spun for 10 min at 6000×g to remove any remaining liquid. Cyanobacterial biomass was added to individual 15 ml falcon tubes as a paste. Biomass was calculated by subtracting the weight of the tube and paste from the weight of the tube only (determined at beginning of experiment).

### Example 5. Greenhouse Testing of Cyanobacterium Treated Corn Seed

A greenhouse trial will be performed with seed from two elite corn hybrids (Monsanto, St. Louis, Mo., USA) with 110 day relative maturities will be coated with three different cyanobacterial mixtures. Mixture A will be the same coating as one of the cyanobacterial mixtures tested above: Nostoc commune UTEX B1621 alone. Mixture B will be a 3:1:1:1 mixture of Nostoc commune UTEX B 1621, Anabaena cylindrica UTEX B 1611, Aulosira bohemensis UTEX B 2947, and Tolypothrix distorta UTEX 424. Mixture C will be a 1:1:1 mixture of Aulosira bohemensis UTEX B 2947, Anabaena cylindrica UTEX B 1611, and Tolypothrix distorta UTEX 424. All cyanobacterial mixtures will be applied at a rate of 4 mg cyanobacterial mixture per seed.

During the trial, seeds coated with Mixture A, Mixture B, Mixture C, and a control with BG 11 media only (no cyanobacteria) seed coating (Table 2) will be grown for 8-10 weeks in an automated greenhouse in Research Triangle Park. Plants will be grown in ˜2 gallon plastic pots in potting soil. They will be watered in four chemical nitrogen regimes: 2 mM, 4 mM, 6 mM and 8 mM. Plants will be shuffled in the greenhouse daily to remove position effects. Physiological parameters that include, but are not limited to: chlorophyll content, biomass, stem diameter and leaf formation will be measured every 24 hours. It is anticipated that seeds coated with Mixture A, Mixture B, or Mixture C will provide corn plants with improved growth characteristics when compared to corn plants obtained from control seeds with BG 11 seed coating.

TABLE 5
Corn Plant Treatments ¹

| Nitrogen (mM) | | | |
|---|---|---|---|
| 2 | 4 | 6 | 8 |

| | | |
|---|---|---|
| Mixture | Number of Plants Tested | Total |

Corn Variety 1

| | | | | | |
|---|---|---|---|---|---|
| A | 20 | 20 | 20 | 20 | 80 |
| B | 20 | 20 | 20 | 20 | 80 |
| C | 20 | 20 | 20 | 20 | 80 |
| Control | 52 | 52 | 52 | 52 | 208 |

Corn Variety 2

| | | | | | |
|---|---|---|---|---|---|
| A | 20 | 20 | 20 | 20 | 80 |
| B | 20 | 20 | 20 | 20 | 80 |
| C | 20 | 20 | 20 | 20 | 80 |
| Control | 52 | 52 | 52 | 52 | 208 |

¹ Table 5 shows the Mixture A, B, C treatments down the left side, nitrogen dosages along the top (mM), and number of plants tested with each Mixture and at each dosage. A total of 416 plants are included in this experiment.

In addition to the greenhouse trial, seeds coated with Mixture A, Mixture B, Mixture C, and no cyanobacterial mixture will be planted in nutrient-rich gel and grown for 4-12 weeks. The plants will be monitored for changes in root morphology daily using an automated root analysis platform. Changes in root morphology observed will be quantified and reported. Parameters measured include, but are not limited to: root hair formation, lateral root frequency, root biomass, and root depth. It is anticipated that seeds coated with Mixture A, Mixture B, and/or Mixture C will provide corn plants with increased root hair formation, lateral root frequency, root biomass, and/or root depth when compared to corn plants obtained from control seeds with BG 11 seed coating.

### Example 6. Cyanobacterial Treatment of Rice and Measurement of Biomass Accumulation and Total Nitrogen Uptake in Green House Studies

All rice experiments were carried out at the University of Arkansas Agriculture Extension. Treatments were blinded. Two varieties of rice, CL 152 (Pureline) and XL 753 (hybrid) were planted in a greenhouse. The plants were treated with topical application of two concentrations (equating to 500 g/acre and 1000 g/acre) of cyanobacterial mixture which consisted of a 3:1:1:1 mixture by weight of Nostoc commune UTEX B 1621, Aulosira bohemensis UTEX B 2947, Anabaena cylindrica UTEX B 1611, and Tolypothrix distorta UTEX 424 respectively. A 3:1:1:1 mixture of Nostoc commune UTEX B 1621, Aulosira bohemensis UTEX B 2947, Anabaena cylindrica UTEX B 1611, and Tolypothrix distorta UTEX 424 respectively were maintained and harvested essentially as described in Example 1. Plants were fertilized with three levels of chemical nitrogen (0, 75 and 150 lbs/acre) in the form of Agrotain™ (Koch Agronomic Services, LLC, Wichita, Kans., USA) treated urea with NBPT urease inhibitor. Plants with no added cyanobacteria served as the control. Plants were harvested at half heading and dried. Total nitrogen uptake and biomass accumulation were measured.

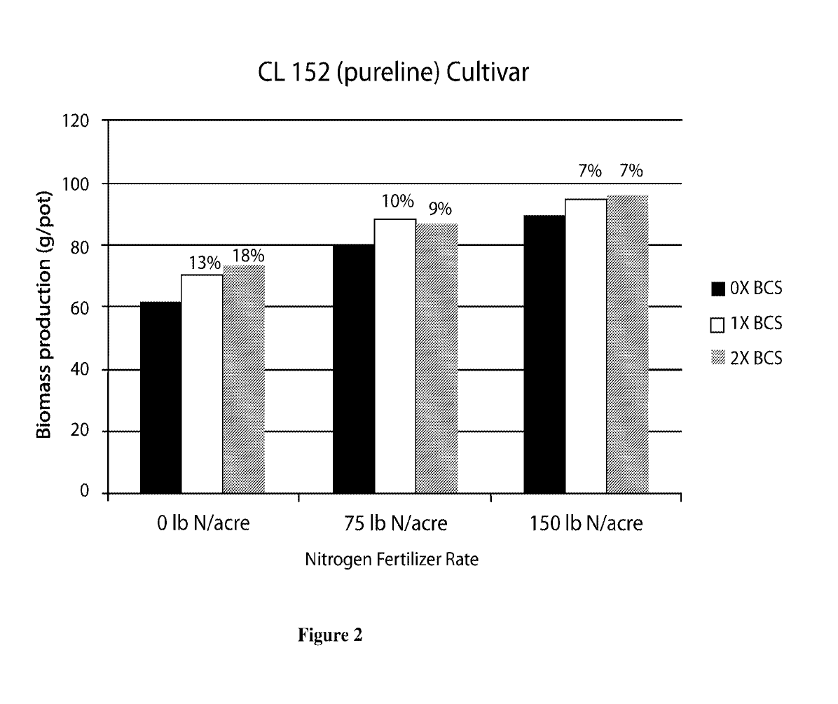

Across both rice varieties and all chemical nitrogen fertilizer rates, statistically significant improvements in biomass accumulation and nitrogen uptake were observed (FIG. 2, shows biomass accumulation in the Pureline). There was no statistical significance between the two cyanobacteria application rates, meaning they were both effective in improving biomass.

### Example 7. Cyanobacterial Treatment of Rice by Treatment of Flooded Fields or by Seed Treatment in Field Tests

A 3:1:1:1 mixture of Nostoc commune UTEX B 1621, Aulosira bohemensis UTEX B 2947, Anabaena cylindrica UTEX B 1611, and Tolypothrix distorta UTEX 424 respectively were maintained and harvested essentially as described in Example 1. Biomass was applied to rice plants at the time of permanent flood establishment by pouring 5 containers containing ⅕ of the total dose into the field with care being taken to distribute the applications as evenly as possible. The plots were 16′×5′. The cyanobacterial mixture was applied evenly at ˜3 foot intervals across a 16 foot plot.

Rice seed coating was carried out using a 3:1:1:1 mixture of Nostoc commune UTEX B 1621, Aulosira bohemensis UTEX B 2947, Anabaena cylindrica UTEX B 1611, and Tolypothrix distorta UTEX 424 respectively, harvested as above. The cyanobacterial mixture was then diluted to a working concentration of 0.2 g biomass/ml liquid. Seed coating was carried out in a in a Hege™ 11 Seed Coater. A total of 1 kg of rice seed was coated with the cyanobacterial biomass mixture. Rice is planted at 100 lbs/acre, so 1 kg of rice seed covers 0.022 acres. With a desired coating rate of 500 g cyanobacterial mixture per acre and a cyanobacterial mixture diluted to 0.2 g biomass/ml cyanobacterial mixture, a total of 55 ml cyanobacterial mixture was used. This cyanobacterial mixture was applied in ˜20 ml aliquots, and seeds were allowed to dry before applying additional cyanobacterial mixture. After all coating was performed, seeds were spread out into a single layer and allowed to dry overnight at room temperature in a well-ventilated area.

One rice cultivar (Pureline CL 152) was grown in three chemical nitrogen fertilizer rates (0, 75, and 150 lbs/acre) in 80 ft² dam-blocked plots. Fertilizer was applied as AgroTain™ (Koch Agronomic Services, LLC, Wichita, Kans., USA) treated urea with NBPT Urease Inhibitor. Rice was planted on Apr. 24, 2014 at the Pinetree Research Station near Colt, Ark., USA using direct-seeding, delayed flood techniques. When the permanent flood was established the topical applications of cyanobacterial mixtures were added to the 0 and 75 lbs/acre nitrogen plots. Five topical cyanobacterial application rates were used (0, 50, 250, 500, and 1000 g/acre cyanobacterial mixture) for each nitrogen application rate. For the seed coating application, the cyanobacteria were applied at the time of coating and were grown in 0, 75, and 150 lbs/acre nitrogen. It is estimated that the seed coating application was equivalent to a topical application rate of about 500 g/acre of the cyanobacterial mixture. Upon reaching full maturity, rice was harvested and yield and total nitrogen uptake were measured. Total above ground biomass was collected from a 1 m section of the first bordered row at 50% heading to determine total N uptake based on published methods (Norman et al. Seasonal accumulation and partitioning of nitrogen-15 in rice. Soil Sci. Soc. Am. J. 56:1521-1527. 1992). Following maturity, plots were allowed to dry to <20% moisture and were harvested using a plot combine. Plot weights and moistures were adjusted to 12.5% and extrapolated to provide a yield estimate. Comparison of cyanobacteria rates within preflood N rates was completed to determine the potential benefit of cyanobacteria under various N management strategies. Statistical analysis was conducted using a standard ANOVA and means were separated using Fisher's protected LSD at the α=0.05 level where appropriate. Table 6 shows two application methods tested (Column 1), and various cyanobacteria application rates (Column 2). Application method A is topical application in the field at time of flood establishment. Application method B was a seed coating applied to the seeds as outlined above. Topical application rates below 500 g/acre were tested for application method A, but improvements observed there were not statistically significant. The field test results are summarized in Table 7. Yield is reported in absolute terms (Column 3) and relative to the baseline (Column 4). The most dramatic yield effect (a 22.2% increase) was observed with the seed treatment method. Increases in yield were positively correlated to Total Nitrogen Uptake.

TABLE 6
Field test results of topical application (Method A) and seed treatment
(Method B)

| | N Application Rate (lbs/acre) | | | | | |
|---|---|---|---|---|---|---|
| | 0 | | 75 | | 150 | |
| Mixture | Total N | | Total N | | Total N | |
| Application | Uptake | Yield | Uptake | Yield | Uptake | Yield |
| Rate (g/acre) | (lb N/acre) | (bu/acre) | (lb N/acre) | (bu/acre) | (lb N/acre) | (bu/acre) |

Method A (Topical Treatment)

| | | | | | | |
|---|---|---|---|---|---|---|
| 0 | 37.8 C | 82.5 C | 64.1 A | 133.8 A | 103.8 A | 151.5 A |
| 50 | 41.9 C | 85.0 BC | 65.6 A | 129.0 A | n/a | n/a |
| 250 | 40.2 C | 82.8 BC | 72.5 A | 128.8 A | n/a | n/a |
| 500 | 41.8 C | 87.3 BC | 62.4 A | 133.0 A | n/a | n/a |
| 1000 | 49.3 B | 90.3 B | 66.1 A | 133.3 A | n/a | n/a |

Method B (Seed Treatment)

| | | | | | | |
|---|---|---|---|---|---|---|
| 500 | 60.0 A | 100.8 A | 71.3 A | 134.8 A | 103.2 A | 152.3 A |

TABLE 7
Summary of Field Test Data

| | BSC | | | Total N | |
|---|---|---|---|---|---|
| | Rate | Yield | % | Uptake | % |
| Method | (g/acre) | (bu/ac) | Increase | (lb N/ac) | Increase |
| A | 0 | 82.5 | 0 | 37.8 | 0 |
| A | 500 | 87.3 | 5.8% | 41.8 | 7.9% |
| A | 1000 | 90.3 | 9.5% | 49.3 | 30.4% |
| B | 500 | 100.8 | 22.2% | 60.0 | 58.7% |

### Example 8. Identification of Active Components of Cyanobacterial Mixture in Greenhouse Experiments

A greenhouse trial in rice was started in which the concentration of the individual components that comprise the cyanobacterial mixture used in the previous examples (Nostoc commune UTEX B 1621, Aulosira bohemensis UTEX B 2947, Anabaena cylindrica UTEX B 1611, and Tolypothrix distorta UTEX 424) were provided in different combinations and at various concentrations as shown in Table 8.

TABLE 8
Cyanobacteria Product treatment list containing the composition of the
individual bacteria within each treatment and the total biomass of
cyanobacteria applied to each treatment.

| | | | | | | | Total |
|---|---|---|---|---|---|---|---|
| | | Nostoc | Aulosira | Anabaena | Tolypothrix | Nitrogen | Biomass |
| Set | Treatment | g/pot | g/pot | g/pot | g/pot | g/pot | g/pot |
| Positive | 1 | 0.049 | 0.014 | 0.014 | 0.014 | 0.94 | 0.091 |
| Control | | | | | | | |
| Positive | 2 | 0.049 | 0.014 | 0.014 | 0.014 | 0 | 0.091 |
| Control | | | | | | | |
| Positive | 3 | 0.007 | 0.002 | 0.002 | 0.002 | 0.94 | 0.013 |
| Control | | | | | | | |
| Positive | 4 | 0.007 | 0.002 | 0.002 | 0.002 | 0 | 0.013 |
| Control | | | | | | | |
| Negative | 5 | 0 | 0 | 0 | 0 | 0.94 | 0 |
| Control | | | | | | | |
| Negative | 6 | 0 | 0 | 0 | 0 | 0 | 0 |
| Control | | | | | | | |
| N-3 | 7 | 0.049 | 0 | 0 | 0 | 0.94 | 0.049 |
| N-3 | 8 | 0 | 0.014 | 0 | 0 | 0.94 | 0.014 |
| N-3 | 9 | 0 | 0 | 0.014 | 0 | 0.94 | 0.014 |
| N-3 | 10 | 0 | 0 | 0 | 0.014 | 0.94 | 0.014 |
| N-3 | 11 | 0.049 | 0 | 0 | 0 | 0 | 0.049 |
| N-3 | 12 | 0 | 0.014 | 0 | 0 | 0 | 0.014 |
| N-3 | 13 | 0 | 0 | 0.014 | 0 | 0 | 0.014 |
| N-3 | 14 | 0 | 0 | 0 | 0.014 | 0 | 0.014 |
| N-2 | 15 | 0.049 | 0.014 | 0 | 0 | 0.94 | 0.063 |
| N-2 | 16 | 0.049 | 0 | 0.014 | 0 | 0.94 | 0.063 |
| N-2 | 17 | 0.049 | 0 | 0 | 0.014 | 0.94 | 0.063 |
| N-2 | 18 | 0.049 | 0.014 | 0 | 0 | 0 | 0.063 |
| N-2 | 19 | 0.049 | 0 | 0.014 | 0 | 0 | 0.063 |
| N-2 | 20 | 0.049 | 0 | 0 | 0.014 | 0 | 0.063 |
| N-2 | 21 | 0 | 0.014 | 0.014 | 0 | 0.94 | 0.028 |
| N-2 | 22 | 0 | 0.014 | 0 | 0.014 | 0.94 | 0.028 |
| N-2 | 23 | 0 | 0.014 | 0.014 | 0 | 0 | 0.028 |
| N-2 | 24 | 0 | 0.014 | 0 | 0.014 | 0 | 0.028 |
| N-2 | 25 | 0 | 0 | 0.014 | 0.014 | 0.94 | 0.028 |
| N-2 | 26 | 0 | 0 | 0.014 | 0.014 | 0 | 0.028 |
| N-1 | 27 | 0.049 | 0.014 | 0.014 | 0 | 0.94 | 0.077 |
| N-1 | 28 | 0.049 | 0.014 | 0 | 0.014 | 0.94 | 0.077 |
| N-1 | 29 | 0.049 | 0 | 0.014 | 0.014 | 0.94 | 0.077 |
| N-1 | 30 | 0.049 | 0.014 | 0.014 | 0 | 0 | 0.077 |
| N-1 | 31 | 0.049 | 0.014 | 0 | 0.014 | 0 | 0.077 |
| N-1 | 32 | 0.049 | 0 | 0.014 | 0.014 | 0 | 0.077 |
| N-1 | 33 | 0 | 0.014 | 0.014 | 0.014 | 0.94 | 0.042 |
| N-1 | 34 | 0 | 0.014 | 0.014 | 0.014 | 0 | 0.042 |

Cultures were maintained and harvested as above, and cyanobacterial mixtures were prepared as above. One rice variety was planted (Pureline CL 152), as results obtained from Greenhouse Trial I (Example 6) showed similar performance for both rice varieties. Two nitrogen application rates (0 and 75 lbs/acre) were applied in the form of Agrotain™ (Koch Agronomic Services, LLC, Wichita, Kans., USA) treated urea with NBPT urease inhibitor. Trials were established in the greenhouse in June of 2014 in sealed pots containing a Captina silt loam soil obtained from the Arkansas Agricultural Research and Extension Center near Fayetteville, Ark. Soils were amended with P and K fertilizer to ensure that no nutrients other than N were limiting rice growth. The cultivar used in the trial was CL 152, which represents one of the more commonly grown pureline cultivars grown in Arkansas. Pots planted to CL 152 were thinned to six plants per pot which would represent the standard seeding rate for this cultivar. Nitrogen rates included in the trial were an untreated control and 75 lb N/acre (suboptimal). Fertilizer was applied to a dry soil and a permanent flood was established when the rice had reached the 4-leaf growth stage. Roughly 3 days following establishment of a permanent flood, cyanobacteria were applied in rates and combinations outlined in Table 8. The experimental design used for this experiment was a randomized complete block design with a full factorial arrangement of 2 N rates×17 cyanobacteria products replicated 5 times. Rice was allowed to grow until 50% heading at which time the entire above ground biomass was harvested from each pot and dried at 60° C. until the biomass reached a constant weight. Following drying, the biomass was ground to pass through a 2 mm sieve and subjected to dry combustion techniques to determine total N concentration. Total N uptake was evaluated using a simple one-way ANOVA to determine if there was a significant influence of cyanobacteria product within a N rate. Biomass accumulation was measured when plants reached 50% heading. Mixtures containing Aulosira and Anabaena (Table 5; treatment 23), Aulosira alone (Table 5; treatment 12), and Aulosira and Tolypothrix (Table 5; treatment 24), showed the highest improvement in nitrogen uptake when no nitrogen was provided. Treatment with Nostoc and Tolypothrix (Table 5; treatment 20) also provided a statistically significant improvement in nitrogen uptake relative to the negative control.

In the suboptimal N treatment, where 75 lb N/acre was applied, the differences between cyanobacteria products and the untreated control were not as great as when no N fertilizer was added. The LSD0.05 to compare cyanobacteria rates within the 75 lb N/acre rate was 0.04569 g N/pot. However, six of the cyanobacteria treatment combinations exhibited total N uptake values greater than that of the untreated control indicating that even under conditions where N fertilizer was applied these cyanobacteria products were able to continue supplying N to the rice plant thereby increasing its overall total N uptake. These products were treatments 8, 22, 33, 7, 17 and 10. Of those treatments, 8 (Aulosira alone) and 22 (Aulosira and Tolypothrix) had the highest total N uptake of any cyanobacteria treatment.

Although this trial was not taken to yield, this data would indicate the potential for yield increases due to the application of certain sub-combinations of the 4 strain cyanobacteria cyanobacterial mixture (Nostoc commune UTEX B 1621, Aulosira bohemensis UTEX B 2947, Anabaena cylindrica UTEX B 1611, and Tolypothrix distorta UTEX 424), especially where N application rates were suboptimal.

TABLE 9
Total N Uptake with various cyanobacterial mixture compositions

| | | | | | | | | |
|---|---|---|---|---|---|---|---|---|
| | Total N | Statistical | | Control | | | | |
| Treatment | Uptake | Grouping | Inoculants | Type | Nos¹ | Aul² | Ana³ | Toly⁴ |

Nitrogen Rate 0 lbs/acre

| | | | | | | | | |
|---|---|---|---|---|---|---|---|---|
| 23 | 0.0966 | a | Aul, Ana | | | X | x | |
| 12 | 0.0849 | ab | Aul | | | X | | |
| 24 | 0.0806 | abc | Aul, Toly | | | X | | x |
| 20 | 0.0803 | bcd | Nos, Toly | | x | | | x |
| 11 | 0.0763 | bcde | Nos | | x | | | |
| 19 | 0.0727 | bcde | Nos, Ana | | x | | x | |
| 14 | 0.0684 | cdef | Toly | | | | | x |
| 4 | 0.0667 | cdef | Nos, Aul, | Positive | x | X | x | x |
| | | | Ana, Toly | Control | | | | |
| 34 | 0.0666 | cdef | Aul, Ana, | | | X | x | x |
| | | | Toly | | | | | |
| 18 | 0.0661 | def | Nos, Aul | | x | X | | |
| 30 | 0.0624 | def | Nos, Aul, | | x | X | x | |
| | | | Ana | | | | | |
| 31 | 0.0624 | def | Nos, Aul, | | x | X | | x |
| | | | Toly | | | | | |
| 13 | 0.061 | def | Ana | | | | x | |
| 6 | 0.061 | def | None | Negative | | | | |
| | | | | Control | | | | |
| 26 | 0.06 | def | Ana, Toly | | | | x | x |
| 32 | 0.0565 | ef | Nos, Ana, | | x | | x | x |
| | | | Toly | | | | | |
| 2 | 0.0518 | f | Nos, Aul, | Positive | x | X | x | x |
| | | | Ana, Toly | Control | | | | |
| | | | | (4x) | | | | |

Nitrogen Rate 75 lbs/acre

| | | | | | | | |
|---|---|---|---|---|---|---|---|
| 8 | 0.2128 | a | Aul | | X | | |
| 22 | 0.2064 | a | Aul, Toly | | X | | x |
| 33 | 0.2014 | ab | Aul, Ana, | | X | x | x |
| | | | Toly | | | | |
| 7 | 0.2014 | ab | Nos | x | | | |
| 17 | 0.1938 | abc | Nos, Toly | x | | | x |
| 10 | 0.193 | abc | Toly | | | | x |
| 25 | 0.1917 | abcd | Ana, Toly | | | x | x |

Nitrogen Rate 0 lbs/acre

| | | | | | | | | |
|---|---|---|---|---|---|---|---|---|
| 28 | 0.1914 | abcd | Nos, Aul, | | x | X | | x |
| | | | Toly | | | | | |
| 21 | 0.1906 | abcd | Aul, Ana | | | X | x | |
| 15 | 0.1892 | abcd | Nos, Aul | | x | X | | |
| 5 | 0.188 | abcd | None | Negative | | | | |
| | | | | Control | | | | |
| 27 | 0.1879 | abcd | Nos, Aul, | | x | X | x | |
| | | | Ana | | | | | |
| 3 | 0.1729 | abcd | Nos, Aul, | Positive | x | X | x | x |
| | | | Ana, Toly | Control | | | | |
| | | | | (1x) | | | | |
| 9 | 0.1655 | abcd | Ana | | | | x | |
| 16 | 0.1538 | bcd | Nos, Ana | | x | | x | |
| 29 | 0.1503 | cd | Nos, Ana, | | x | | x | x |
| | | | Toly | | | | | |

Key:
Nos¹ = Nostoc commune UTEX B 1621;
Aul² = Aulosira bohemensis UTEX B 2947;
Ana³ = Anabaena cylindrica UTEX B 1611; and
Toly⁴ = Tolypothrix distorta UTEX 424.

### Example 9. Greenhouse Testing of Cyanobacterium Treated Corn Seed

A greenhouse trial was performed with seed from two elite corn hybrids (Monsanto, St. Louis, Mo., USA) with 110 day relative maturities. These were coated with three different cyanobacterial mixtures. Mixture A was the same coating as one of the cyanobacterial mixtures tested above: Nostoc commune UTEX B1621 alone. Mixture B was a 3:1:1:1 mixture of Nostoc commune UTEX B 1621, Anabaena cylindrica UTEX B 1611, Aulosira bohemensis UTEX B 2947, and Tolypothrix distorta UTEX 424. Mixture C was a 1:1:1 mixture of Aulosira bohemensis UTEX B 2947, Anabaena cylindrica UTEX B 1611, and Tolypothrix distorta UTEX 424. All cyanobacterial mixtures were applied at a rate of 4 mg cyanobacterial mixture per seed.

During the trial, seeds coated with Mixture A, Mixture B, Mixture C, and a control with BG 11 media only (no cyanobacteria) seed coating (Table 2) will be grown for 10 weeks in an automated greenhouse in Research Triangle Park. Plants were grown in ˜2 gallon plastic pots in potting soil. They were watered and nitrogen fertilizer will be applied at four levels: 2 mM, 4 mM, 6 mM and 8 mM. Plants were shuffled in the greenhouse daily to remove position effects. Physiological parameters that include, but are not limited to: chlorophyll content, biomass, stem diameter and leaf formation were measured every 24 hours. It was anticipated that seeds coated with Mixture A, Mixture B, or Mixture C will provide corn plants with improved growth characteristics when compared to corn plants obtained from control seeds with BG 11 seed coating.

TABLE 10
Treatments, Nitrogen Dosages, and Numbers of Plants Tested

| Nitrogen (mM) | | | |
|---|---|---|---|
| 2 | 4 | 6 | 8 |

| | | |
|---|---|---|
| Mixture | Number of Plants Tested | Total |

Corn Variety 1

| | | | | | |
|---|---|---|---|---|---|
| A | 20 | 20 | 20 | 20 | 80 |
| B | 20 | 20 | 20 | 20 | 80 |
| C | 20 | 20 | 20 | 20 | 80 |
| Control | 52 | 52 | 52 | 52 | 208 |

Corn Variety 2

| | | | | | |
|---|---|---|---|---|---|
| A | 20 | 20 | 20 | 20 | 80 |
| B | 20 | 20 | 20 | 20 | 80 |
| C | 20 | 20 | 20 | 20 | 80 |
| Control | 52 | 52 | 52 | 52 | 208 |

Table 10 shows the Mixture A, B, C treatments down the left side, nitrogen dosages along the top (mM), and number of plants tested with each Mixture and at each dosage. A total of 416 plants are included in this experiment.

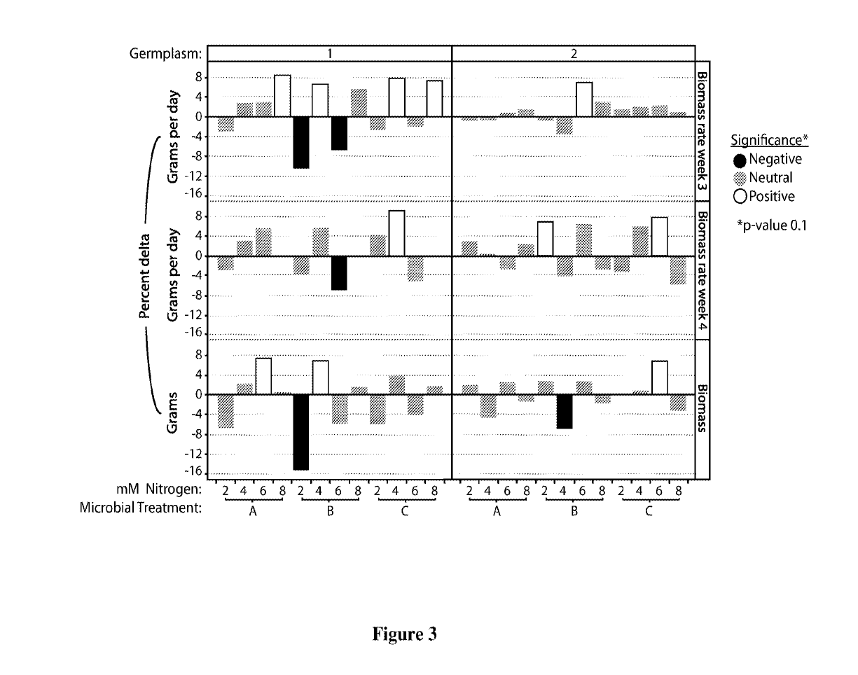

Germination levels for treated seeds were the same as untreated. Untreated plants had the expected response to nitrogen deficiency-lower levels of biomass, more yellowing, lower height, etc. Treated plants in all Mixtures showed more increases in biomass than reductions, as evidenced in FIG. 3.

In addition to the greenhouse trial, seeds coated with Mixture A, Mixture B, Mixture C, and no cyanobacterial mixture were planted in nutrient-rich gel and grown for 8 weeks. The plants were monitored for changes in root morphology daily using an automated root analysis platform. Changes in root morphology observed were quantified and reported. Parameters measured include, but are not limited to: root hair formation, lateral root frequency, root biomass, and root depth. It was anticipated that seeds coated with Mixture A, Mixture B, and/or Mixture C will provide corn plants with increased root hair formation, lateral root frequency, root biomass, and/or root depth when compared to corn plants obtained from control seeds with BG 11 seed coating.

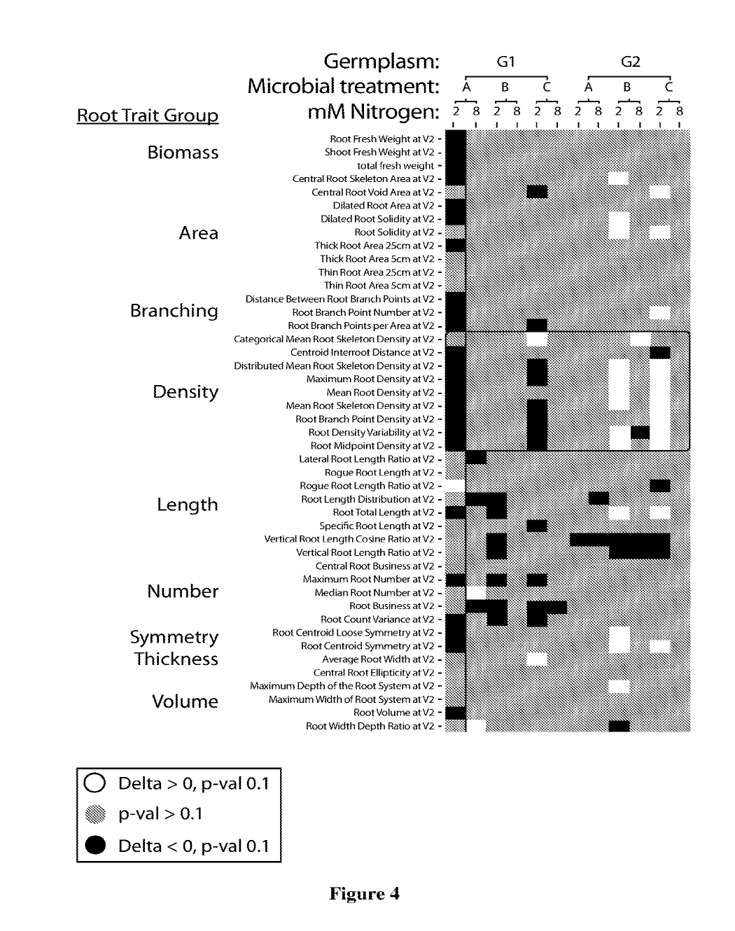

Two different fertilizer levels were tested, 2 mM and 8 mM. Germplasm 2 had the strongest positive response to the treatment, showing increases in biomass, branching, density and symmetry of roots. The results are depicted in FIG. 4.

### Example 10. Cyanobacterial Treatment of Rice and Measurement of Biomass Accumulation and Total Nitrogen Uptake in Green House Studies

Rice experiments were carried out at the University of Arkansas Agriculture Extension at two locations. Treatments were blinded. One variety of rice, CL 152, a pureline was planted in a field trial. The plants were treated with topical application of three concentrations (equating to 250 g/acre, 500 g/acre and 1000 g/acre) of cyanobacterial mixture which consisted of a 2:1 mixture by weight of Aulosira bohemensis UTEX B 2947 and Tolypothrix distorta UTEX 424, respectively. Cyanobacteria were maintained and harvested essentially as described in Example 1. Plants were fertilized with two levels of chemical nitrogen (165 lbs/acre and 186 lbs/acre) in the form of Agrotain™ (Koch Agronomic Services, LLC, Wichita, Kans., USA) treated urea with NBPT urease inhibitor. A fertilizer rate of 186 lbs/acre represents an optimized level for current agronomic practices in the United States. A fertilizer rate of 165 lbs/acre is achieved by omitting the initial or ‘starter’ application of fertilizer. Plants with no added cyanobacteria served as the control. Plants were harvested at full heading and dried. Total nitrogen uptake and biomass accumulation were measured.

The results of this field trial show that yield at optimal fertilizer and no Cyanobacteria treatment is equal to yield upon addition of Cyanobacteria treatment at the 1000 g/acre dosage and omission of the initial or ‘starter’ application of fertilizer. This was true at both field trial locations. Yield measurement upon addition of Cyanobacteria and omission of initial or ‘starter’ application of fertilizer was actually 1.5% higher than yield at optimal fertilizer and no Cyanobacteria. Table 11 shows the relevant yield data from both locations.

TABLE 11
Yield increase in the presence of full fertilizer, full
fertilizer—starter application, and Cyanobacteria.

| | Location | Location | Percentage |
|---|---|---|---|
| | A Yield | B Yield | Change |
| Nitrogen Application | (bushels/acre) | (bushels/acre) | (average) |
| Full Fertilizer (186 lb N) | 183 | 163 | — |
| Full Fertilizer—Starter | 179 | 152 | −4.46% |
| (165 lb N) | | | |
| 165 lb N + 250 g/acre | 184 | 151 | −3.41% |
| Cyanobacteria | | | |
| 165 lb N + 500 g/acre | 185 | 157 | −1.30% |
| Cyanobacteria | | | |
| 165 lb N + 1000 g/acre | 185 | 166 | +1.46% |
| Cyanobacteria | | | |

### Example 11. Cyanobacterial Treatment of Rice by Treatment of Flooded Fields or by Seed Treatment in Field Tests

A 2:1 mixture of Aulosira bohemensis UTEX B 2947 and Tolypothrix distorta UTEX 424 respectively was maintained and harvested essentially as described in Example 1. These Cyanobacteria were applied to rice seeds at a rate of 1 kg/acre. There were three application methods, 1 kg/acre as a seed coating, ½ kg per acre seed coating +½ kg acre liquid, and 1 kg/acre liquid. The seed coating was applied at the time of planting. The liquid application was applied after transplanting. Hybrid seed MRP 5566 and variety seed MRP 1010 were provided by Maharashtra Hybrid Seed Company, Private Limited of India. The plots were 10 m×5 m and were randomized in a complete block to eliminate variation. All plots were dam blocked to prevent contamination.

Rice seed coating was carried out using a 2:1 mixture of Aulosira bohemensis UTEX B 2947 and Tolypothrix distorta UTEX 424, harvested as above. The cyanobacterial mixture was then diluted to a working concentration of 0.2 g biomass/ml liquid. Seed coating was carried out in plastic bags. A total of 1 kg of rice seed was coated with the cyanobacterial biomass mixture. Transplant plots will be retained until full establishment; the extras can then be used to plug holes; at later stages when plants may be lost to snails or other pests, purple rice transplants should be on hand for those gaps.

The field was ploughed and puddled multiple times until a fine soft puddle is developed. A raised bed of about 1 meter wide and 5 cm height was prepared with drainage for excess water. Paddy seeds were planted using a broadcasting method on the leveled seed bed which was maintained in moist conditions until seeds germinated. Once seedlings were about 2 cm tall, shallow water level was maintained enough to cover the soil in nursery bed.

Broadcasting of seeds was done such that seedlings are not too close to each other and can grow with sufficient distance to flourish. Approximately, 150 sq. m nursery is sufficient to raise seedlings for 1 acre rice field. This will vary slightly depending on plant density in the final field. While preparing seed bed, prior to final puddling/leveling, about 0.5 kg of N, 0.25 Kg P and 0.35 kg K was added per 150 sq. m area.

Seedlings were uprooted by hand by holding 3-4 seedlings in a bunch and pulling them up. If too much mud comes stuck with root, they were rinsed in water to dislodge excess soil. This also helps in separating seedlings during transplanting. Seedlings were carried to main field for transplanting.

Main field was prepared by ploughing, puddling and leveling. Fertilizer at recommended dosage was applied during final soil preparation activity before transplanting. 1-2 seedlings per hill were planted by hand in rows to maintain desired plant to plant and row to row spacing. Lines with 15 cm markings are used while transplanting to maintain proper spacing.

Drying was used as a draining mechanism until a week before harvest, when the field was drained to facilitate entering the field by workers or small machinery. Plant-to-plant distances in previous trials were less than 3 cm. 15 to 20 cm will be maintained between plants for this trial.

The results of the field trial are shown in Table 12. The combination of the seed coating and liquid treatments produced yield increases in comparison to controls that were about 2-fold greater than the yield increases provided by the seed coating alone and about 5-fold higher than the yield increases provided by the liquid treatment alone.

TABLE 12
Field test results of Cyanobacteria application to seeds in field
trial in India.

| Cyanobacterial | Variety Seed | | Hybrid Seed | |
|---|---|---|---|---|
| Application | Yield | % Δ to | Yield | % Δ to |
| Method | (kg/acre) | Control | (kg/acre) | Control |
| Seed Coating | 2259.8 | 13.83% | 2640.0 | 7.30% |
| ½ kg/acre Seed | 2096.2 | 5.59% | 2830.0 | 15.02% |
| Coating | | | | |
| ½ kg/acre Liquid | | | | |
| Liquid | 2112.1 | 6.39% | 2534.4 | 3.00% |
| Control | 1985.2 | — | 2460.5 | — |

The breadth and scope of the present disclosure should not be limited by any of the above-described exemplary embodiments, but should be defined only in accordance with the following claims and their equivalents. As various changes could be made in the above compositions and methods without departing from the scope of the invention, it is intended that all matter contained in the above description and shown in the accompanying drawings shall be interpreted as illustrative and not in a limiting sense.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

1. A treated monocot plant seed comprising a solid coating on at least a portion of an outer surface of the seed, the coating comprising cyanobacteria comprised of an Aulosira species and a Tolypothrix species, wherein either:
(1) the coating is free of an agriculturally acceptable adjuvant and/or an agriculturally acceptable excipient; or
(2) the coating further comprises the adjuvant and/or the excipient in an amount less than 0.09 gram per gram of seed; or
(3) grain yield resulting from the treated monocot plant seed other than rice is at least 2.0% greater than grain yield resulting from the same monocot plant seed that is not treated with the coating when grown under the following greenhouse conditions:
(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) for rice, permanent flood is established at 4-leaf growth stage;
(c) temperature maintained at 25-28° C., 50-70% relative humidity and natural light supplemented with fluorescent light at 35 μmol m⁻² s⁻² photosynthetically active radiation to provide a 14 h photoperiod; and
(d) plant grown to 50% heading then entire above-ground biomass is collected and dried, and total weight and nitrogen concentration can be measured; or
(4) grain yield resulting from the treated rice seed is at least 10.0% greater than grain yield resulting from the same rice seed that is not treated with the coating when grown under the greenhouse conditions; or
(5) the cyanobacteria consists essentially of the Aulosira species and the Tolypothrix species; or
(6) the coating consists essentially of the Aulosira species and the Tolypothrix species; or
(7) the coating comprises the Aulosira species and the Tolypothrix species in a weight ratio of about 5:1 to about 1:5.

2. The treated monocot plant seed of claim 1, wherein the coating is free of an agriculturally acceptable adjuvant or an agriculturally acceptable excipient.

3. The treated monocot plant seed of claim 1, wherein the coating is free of an agriculturally acceptable adjuvant and an agriculturally acceptable excipient.

4. The treated monocot plant seed of claim 1, wherein the coating further comprises the adjuvant and/or the excipient in an amount less than 0.08, 0.07, 0.06, 0.05, 0.04, 0.03, 0.02, or 0.01 gram per gram of seed.

5-11. (canceled)

12. The treated monocot plant seed of claim 1, wherein the coating comprises the adjuvant and/or the excipient in an amount of at least 0.001 gram per gram of seed.

13. The treated monocot plant seed of claim 1, wherein the coating further comprises the cyanobacterium in an amount from about 0.001 g to about 0.15 gram per gram of seed.

14. The treated monocot plant seed of claim 13, wherein the coating further comprises the cyanobacterium in an amount less than 0.08, 0.09, 0.10, 0.11, 0.12, 0.13, or 0.14 gram per gram of seed.

15. (canceled)

16. The treated monocot plant seed of claim 1, wherein at least one of the following:
grain yield resulting from the treated monocot plant seed other than rice seed is at least 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater than grain yield resulting from the same monocot plant seed that is not treated with the coating when grown under the greenhouse conditions; and/or
nitrogen uptake resulting from the treated monocot plant seed is at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater than nitrogen uptake resulting from the same monocot plant seed that is not treated with the coating when grown under the greenhouse conditions; and/or
biomass resulting from the treated monocot plant seed is at least 2, 3, 4, 5, 6, 7, 8, 9, 10, 11, 12, 13, 14, 15, 16, 17, 18, 19, 20, 21, 22, 23, 24, or 25% greater than biomass resulting from the same monocot plant seed that is not treated with the coating when grown under the greenhouse conditions; and/or
lateral root growth, lateral root hair growth, vertical root growth or root branching resulting from the treated monocot plant seed exhibits increased branching as compared to lateral root growth, lateral root hair growth, vertical root growth or root branching resulting from the same monocot plant seed that is not treated with the coating when grown under the greenhouse conditions; and/or
nutrient uptake resulting from the treated monocot plant seed is at least 2, 3, 4, 5, 6, 7, 8, 9, or 10% greater than nutrient uptake resulting from the same monocot plant seed that is not treated with the coating when grown under the greenhouse conditions.

17-23. (canceled)

24. The treated monocot plant seed of claim 16, wherein at least one of the following:
the treated monocot plant seed is rice, and either: grain yield is increased by 10-25%; nitrogen uptake is increased by about 8-70%; or biomass is increased by about 3-25%; and/or
the treated monocot plant seed is wheat or corn, and either: grain yield is increased by about 2-8%; nitrogen uptake is increased by about 5-20%; or biomass is increased by about 2-12%; and/or
plant growth medium in which the plant seed is grown is supplemented with less than 10, 15, 20, 25, 30, 35, 40, 45, 50, 100 or 200 lbs nitrogen fertilizer/acre.

25-29. (canceled)

30. The treated monocot plant seed of claim 24, wherein either: the plant growth medium in which the plant seed is grown is supplemented with at least 0.1 lb nitrogen fertilizer/acre; or the plant growth medium in which the plant seed is grown is not supplemented with nitrogen.

31. (canceled)

32. The treated monocot plant seed of claim 1, wherein the cyanobacteria consists essentially of the Aulosira species and the Tolypothrix species; or
the coating consists essentially of the Aulosira species and the Tolypothrix species; or
the coating comprises the Aulosira species and the Tolypothrix species in a weight ratio of about 5:1 to about 1:5, about 3:1 to about 1:3, about 3:1 to 1.1:1, about 3:1 to about 1.5:1, about 2.5:1 to about 1.5:1, or about 2.2:1 to about 1.8:1.

33-39. (canceled)

40. The treated monocot plant seed of claim 1, wherein:
the monocot plant seed comprises a rice seed, a corn seed, a sorghum seed, a turfgrass seed, a wheat seed, a biofuel crop seed, a forage crop seed, or a millet seed; or
the monocot plant seed comprises the turfgrass seed, and the turfgrass seed comprises a bentgrass, bermudagrass, bluegrass, buffalograss, fescue, or ryegrass seed; or
the monocot plant seed comprises the forage crop seed, and the forage crop seed comprises a hay, ryegrass, or oat seed; or
the monocot plant seed comprises the biofuel crop seed, and the biofuel crop seed comprises a Miscanthus or switchgrass seed.

41-43. (canceled)

44. The treated monocot plant seed of claim 1, wherein at least one of the following:
the Aulosira species is characterized by having a gene encoding a 16S RNA that has at least 95%, 97%, 98%, 99%, 99.5%, 99.7%, 99.9%, or 100% sequence identity across the entire length of SEQ ID NO:1; and/or
the Tolypothrix species is characterized by having a gene encoding a 16S RNA that has at least 95%, 97%, 98%, 99%, 99.5%, 99.7%, 99.9%, or 100% sequence identity across the entire length of SEQ ID NO:4; and/or
the Aulosira species comprises an Aulosira bohemensis UTEX B 2947 culture or variant thereof or wherein the Tolypothrix species comprises a Tolypothrix distorta UTEX 424 culture or a variant thereof; and/or
the Aulosira species comprises an Aulosira bohemensis UTEX B 2947 culture or variant thereof and wherein the Tolypothrix species comprises a Tolypothrix distorta UTEX 424 culture or a variant thereof; and/or
0.0001 milligram to 5 milligram (dry weight) of the Aulosira species and a Tolypothrix species is provided on the seed; and/or
less than 0.0005, 0.001, 0.005, 0.01, 0.02, 0.03, 0.04, 0.05, 0.06, 0.07, 0.08, 0.09, 0.10, 0.11, 0.12, 0.13, 0.14, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, or 4.5 milligram wet or dry weight of the Aulosira species and a Tolypothrix species is provided on the seed; and/or
wherein the agriculturally acceptable adjuvant comprises an insecticide, a fungicide, a safener, a biological fertilizer, a water retention compound, a nutrient retention compound, biochar or a combination thereof; and/or
wherein the agriculturally acceptable adjuvant comprises an insecticide, and the insecticide comprises a carbamate, an organophosphate, a neonicotinoid, a pyrethroid, or a combination thereof; and/or
wherein the agriculturally acceptable adjuvant comprises a neonicotinoid insecticide, and the neonicotinoid insecticide comprises thiamethoxam, imidacloprid, clothianidin, nitenpyram, nithiazine, thiacloprid, or a combination thereof; and/or
the coating contains at least one element or a salt thereof comprising iron, boron, manganese, zinc, molybdenum, copper, cobalt, or a combination thereof; and/or
the Aulosira species and the Tolypothrix species are not associated with the monocot plant seed in nature; and/or
the coating comprises an exogenous exopolysaccharide; and/or
the coating is lyophilized; and/or
the cyanobacteria does not further comprise an Anabaena species, a Nostoc species, or both an Anabaena species and a Nostoc species; and/or
the cyanobacteria does not further comprise Tolypothrix tenuis, Aulosira fertilissima, and a Nostoc species; and/or
the cyanobacteria does not further comprise Tolypothrix tenuis, Aulosira fertilissima, a Nostoc species, and a Cylindrospermum species.

45-58. (canceled)

59. A method of obtaining a plurality of monocot crop plants with improved grain yield, nitrogen uptake, nutrient uptake, biomass, lateral root growth, lateral root hair growth, vertical root growth or root branching, the method comprising: distributing the treated monocot plant seed of claim 1 on a plot of land and cultivating plants grown from the seed on the plot.

60. (canceled)

61. A method of obtaining a monocot plant seed that provides for improved growth in a monocot plant obtained from the seed, the method comprising: applying a composition comprising the cyanobacteria to the at least a portion of an outer surface of the seed and lyophilizing or drying the composition to form the solid coating and obtain the treated monocot plant seed of claim 1.

62. (canceled)

63. A method of improving biomass, grain yield, nutrient uptake, nitrogen uptake, lateral root growth, lateral root hair growth, vertical root growth and/or root branching in a monocot plant, the method comprising:
exposing the monocot plant seed to an effective amount of a composition comprising cyanobacteria comprised of an Aulosira species and a Tolypothrix species, wherein either;
(1) the composition is free of an agriculturally acceptable adjuvant and/or an agriculturally acceptable excipient; or
(2) the composition further comprises the adjuvant and/or the excipient in an amount less than 0.4 kg/hectare; or
(3) the cyanobacteria consists essentially of the Aulosira species and the Tolypothrix species; or
(4) the composition consists essentially of the Aulosira species and the Tolypothrix species; or
(5) the composition comprises the Aulosira species and the Tolypothrix species in a weight ratio of about 5:1 to about 1:5; and
allowing the monocot plant seed to germinate;
wherein either:
(A) grain yield resulting from the treated monocot plant seed other than rice is at least 2.0% greater than grain yield resulting from the same monocot plant seed that is not exposed to the composition when grown under greenhouse conditions; or
(B) grain yield resulting from the treated rice seed is at least 10.0% greater than grain yield resulting from the same rice seed that is not exposed to the composition when grown under the greenhouse conditions; or
(C) nitrogen uptake resulting from the treated monocot plant seed is at least 5% greater than nitrogen uptake resulting from the same monocot plant seed that is not exposed to the composition when grown under the greenhouse conditions; or
(D) biomass resulting from the treated monocot plant seed is at least 2% greater than biomass resulting from the same monocot plant seed that is not exposed to the composition when grown under the greenhouse conditions; or
(E) lateral root growth, lateral root hair growth, vertical root growth or root branching resulting from the treated monocot plant seed exhibits increased branching as compared to lateral root growth, lateral root hair growth, vertical root growth or root branching resulting from the same monocot plant seed that is not exposed to the composition when grown under the greenhouse conditions; or
(F) nutrient uptake resulting from the treated monocot plant seed is at least 2% greater than nutrient uptake resulting from the same monocot plant seed that is not exposed to the composition when grown under the greenhouse conditions;
wherein the greenhouse conditions are as follows:
(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) for rice, permanent flood is established at 4-leaf growth stage;
(c) temperature maintained at 25-28° C., 50-70% relative humidity and natural light supplemented with fluorescent light at 35 μmol m⁻² s⁻² photosynthetically active radiation to provide a 14 h photoperiod; and
(d) plant grown to 50% heading then entire above-ground biomass is collected and dried, and total weight and nitrogen concentration can be measured.

64-111. (canceled)

112. A treated particle comprising a solid coating on at least a portion of an outer surface of the particle, the coating comprising cyanobacteria comprised of an Aulosira species, and the particle comprising a fertilizer granule, biological fertilizer, water or nutrient retention device, or a biochar particle.

113-133. (canceled)

134. A method of binding an agriculturally acceptable adjuvant and/or an agriculturally acceptable excipient to a particle, the method comprising: applying a composition comprising the cyanobacteria and the adjuvant and/or excipient to the at least a portion of an outer surface of the particle and lyophilizing or drying the composition to form the solid coating and obtain the treated particle of claim 112.

135. A method of obtaining a plurality of plants comprising: distributing the treated particles of claim 112 on a plot of land; planting the plot with seeds or plants; and cultivating plants grown on the plot.

136. (canceled)

137. A method of obtaining a treated particle comprising:
applying a composition comprising the cyanobacteria to the at least a portion of an outer surface of the particle and lyophilizing or drying the composition to form the solid coating and obtain the treated particle of claim 112.

138-142. (canceled)
